(19) United States
(12) Patent Application Publication
(10) Pub. No.: US 2023/0279054 A1
(43) Pub. Date: Sep. 7, 2023

(54) DE NOVO DESIGNED MIXED CHIRALITY PEPTIDE MACROCYCLES WITH INTERNAL SYMMETRY

(71) Applicant: University of Washington, Seattle, WA (US)

(72) Inventors: Vikram K. MULLIGAN, Seattle, WA (US); Christine KANG, Seattle, WA (US); Timothy CRAVEN, Seattle, WA (US); David BAKER, Seattle, WA (US)

(21) Appl. No.: 18/040,746

(22) PCT Filed: Sep. 23, 2021
(86) PCT No.: PCT/US2021/051633

§ 371 Date: Feb. 6, 2023

Related U.S. Application Data
(60) Provisional application No. 63/083,444, filed on Sep. 25, 2020.

(51) Int. Cl. C07K 7/64

(52) U.S. Cl. CPC C07K 7/64

(57) ABSTRACT

The disclosure provides polypeptide comprising or consisting of an amino acid sequence at least 66%, 70%, 75%, 80%, 82%, 84%, 86%, 88%, 90%, 91%, 92%, 93%, 94%, 95%, 96%, 97%, 98%, 99%, or 100% identical to the amino acid sequence selected from the group consisting of SEQ ID NO: 1-91 as shown in Table I, wherein: (a) amino acid residues in upper case are L amino acids, and residues in lower case are D amino acids; (b) X is 2-aminoisobutyric acid (ATB); (c) no amino acid changes at proline or AIB e residues in the reference peptide are permitted; and (d) any amino acid changes must maintain chirality relative to the reference peptide.

### CROSS REFERENCE

This application claims priority to U.S. Provisional Application Ser. No. 63/083,444 filed Sep. 25, 2020, incorporated by reference herein in its entirety.

### FEDERAL FUNDING STATEMENT

This invention was made with government support under Grant No. HDTRA 1-19-1-0003, awarded by the Defense Threat Reduction Agency. The government has certain rights in the invention.

### SEQUENCE LISTING STATEMENT

A computer readable form of the Sequence Listing is filed with this application by electronic submission and is incorporated into this application by reference in its entirety. The Sequence Listing is contained in the file created on Sep. 14, 2021 having the file name “20-1325-WO-SeqList_ST25.txt” and is 41 kb in size.

### BACKGROUND

Cyclic symmetry is frequent in protein and peptide homo-oligomers, but extremely rare within a single chain, as it is not compatible with free N and C termini. The ability to design symmetric, well-folded polypeptide macrocycles would open up new avenues for both therapeutic design and for bounded and unbounded nanomaterial design.

### SUMMARY

In one aspect, the disclosure provides polypeptide comprising or consisting of an amino acid sequence at least 66%, 70%, 75%, 80%, 82%, 84%, 86%, 88%, 90%, 91%, 92%, 93%, 94%, 95%, 96%, 97%, 98%, 99%, or 100% identical to the amino acid sequence selected from the group consisting of SEQ ID NO: 1-91 as shown in Table 1, wherein:
(a) amino acid residues in upper case are L amino acids, and residues in lower case are D amino acids;
(b) X is 2-aminoisobutyric acid (AIB);
(c) no amino acid changes at proline or AIB residues in the reference peptide are permitted; and
(d) any amino acid changes must maintain chirality relative to the reference peptide.

In one embodiment, no proline or AIB residues may be added by amino acid change relative to the reference polypeptide. In another embodiment, the polypeptide has C2 symmetry, and comprises an amino acid sequence at least 66%, 70%, 75%, 80%, 82%, 84%, 86%, 88%, 90%, 91%, 92%, 93%, 94%, 95%, 96%, 97%, 98%, 99%, or 100% identical to the amino acid sequence selected from the group consisting of SEQ ID NO:1-8. In a further embodiment, the polypeptide has S2 symmetry, and comprises an amino acid sequence at least 66%, 70%, 75%, 80%, 82%, 84%, 86%, 88%, 90%, 91%, 92%, 93%, 94%, 95%, 96%, 97%, 98%, 99%, or 100% identical to the amino acid sequence selected from the group consisting of SEQ ID NO: 9-14. In one embodiment, the polypeptide has C3 symmetry, and comprises an amino acid sequence at least 66%, 70%, 75%, 80%, 82%, 84%, 86%, 88%, 90%, 91%, 92%, 93%, 94%, 95%, 96%, 97%, 98%, 99%, or 100% identical to the amino acid sequence selected from the group consisting of SEQ ID NO:15-72. In one such embodiment, the first residue of the asymmetric unit is L-Proline and the third residue of the asymmetric unit is L-Aspartic acid, the 2nd residue can be any non-glycine, non-proline, non-AIB L-amino acid.

In another embodiment, the polypeptide has C4 symmetry, and comprises an amino acid sequence at least 66%, 70%, 75%, 80%, 82%, 84%, 86%, 88%, 90%, 91%, 92%, 93%, 94%, 95%, 96%, 97%, 98%, 99%, or 100% identical to the amino acid sequence selected from the group consisting of SEQ ID NO:73-76. In one such embodiment, the polypeptide is bound to a metal ion, including but not limited to Zn2⁺.

In one embodiment, the polypeptide has C5 symmetry, and comprises an amino acid sequence at least 66%, 70%, 75%, 80%, 82%, 84%, 86%, 88%, 90%, 91%, 92%, 93%, 94%, 95%, 96%, 97%, 98%, 99%, or 100% identical to the amino acid sequence selected from the group consisting of SEQ ID NO:77-90. In another embodiment, the polypeptide has S4 symmetry, and comprises an amino acid sequence at least 66%, 70%, 75%, 80%, 82%, 84%, 86%, 88%, 90%, 91%, 92%, 93%, 94%, 95%, 96%, 97%, 98%, 99%, or 100% identical to the amino acid sequence of SEQ ID NO:91.

In a further embodiment, the polypeptide is conjugated to one or more additional components, including, but not limited to detectable tags, small molecules, radioactive agents, antibodies, polyethylene glycol, therapeutic moieties, and diagnostic moieties.

The disclosure also provides methods for use of the polypeptide of any embodiment, including but not limited to those uses described herein, and assembling them together with metals to form super molecular crystals. The disclosure further provides methods for designing mixed chirality peptide macrocycles with internal symmetry according to any embodiment or combination of embodiments described herein.

### FIGURE LEGENDS

FIG. 1. Designed peptides with cyclic (C2 or C3) symmetries. Columns: (A) Computer models produced by Rosetta™ design. Backbone-backbone, backbone-sidechain, and sidechain-sidechain hydrogen bonds are shown. The C2 or C3 symmetry axis is shown as a black rod. Hand icons depict the symmetry. (B) Designed amino acid sequences and Ramachandran bin strings. C2 symmetry and C3 symmetry designs are shown, indicating different repeating units. (C) Computed energy landscape of design, in which each point represents a structure prediction trajectory, with computed energy plotted against RMSD to the designed structure. (D) Ramachandran map representation of the designed structures (grey points) compared to the experimentally-determined structures (points by symmetry lobe, as in column B). Grey numbers indicate sequence positions, and curved arrows show the progression through the sequence. Grey ovals group the designed and observed backbone angles, as a guide to the eye. In the case of peptide C2-1, three residues, indicated with numbers, showed considerable deviation in ϕ (horizontal dashed lines) or ψ (vertical dashed line). (E) Structures determined by x-ray crystallography. (F) Overlay of the designed model with the x-ray crystal structure. Symmetric lobes are shown matching sequences in column B. Side-chains other than those of AIB or proline are omitted for clarity. Rows: (I) Peptide C2-1. The backbone heavy-atom RMSD between crystal structure and design was 1 Å, mainly due to shifts in ψ of residue 1 and ϕ of residue 2 (dashed lines in column D and arrows in column F) which together rotated the amide bond between residues 1 and 2 by 180°, and in ϕ of residue 8, which reoriented a backbone carbonyl (arrow) in the absence of the hydrogen bond that would have been donated to it by the rotated amide proton. Despite these changes, much of the structure overlaid well on the design. (II and III) Peptides C3-2 and C3-3, which shared a common backbone configuration. Crystal contacts resulted in somewhat different conformations of polar side-chains, but the backbone heavy-atom RMSDs to the designs were 0.5 Å and 0.3 Å, respectively, yielding near-perfect alignment in both cases (column E).

FIG. 2. Designed peptides with S2 improper rotational symmetry. Unless noted otherwise, columns are as in FIG. 1. The symmetry is illustrated with left and right hands in icons in column A. Centers of inversion are shown as black spheres in columns A and E. In column D, blue ovals indicate the first and second symmetry lobes, respectively, which are related to one another by mirroring (a 180° rotation about the origin in Ramachandran space). (I through VI) Peptides S2-1 through S2-6. In each of peptides S2-4 and S2-5, two designed backbone-backbone hydrogen bonds were observed to be replaced with a bridging water molecule in the crystal structure (dashed lines in column E).

FIG. 3. A designed biconformational metal-binding polypeptide with S4 improper rotational symmetry. (A) Design model of S4-1 polypeptide. Backbone hydrogen bonds are shown as dashed lines, the central bound zinc atom, as a dark grey sphere, polar side-chains, apolar side-chains, and backbone atoms as sticks. Solvent-exposed glutamate, glutamine, and lysine side-chains are omitted for clarity. In this and subsequent panels, the S4 symmetry axis is shown as a black line. The icon adjacent to letter illustrates the symmetry. (B) Sequence and expected Ramachandran bins for each residue in the metal-bound (“Holo bin”) and metal-free (“Apo bin”) states. (C) Polypeptide S4-1 titration into PAR₂Zn solution to measure metal affinity by competition. Based on the known affinity of PAR for zinc, the measured affinity of the S4-1 polypeptide for zinc was 0.32 nM (curve of best fit). (D) X-ray crystal structure of zinc-bound S4-1 polypeptide. The (i+3 →i)3₁₀ helix hydrogen bond pattern that was designed was observed. (E) Overlay of S4-1 design and crystal structure. The backbone heavy-atom RMSD was 0.3 Å, and metal-binding histidine side-chains and surface-exposed leucine side-chains showed excellent agreement to the design. (F) S4-symmetric lowest-energy predicted alternative structure with RMSD greater than 2 Å from the design (arrow in panel G) in the absence of zinc. Although only one glutamine residue per lobe undergoes a major conformational change, this allows inversion of the structure, so that apolar side-chains are now buried. (G) Computationally predicted energy as a function of RMSD to the designed conformation. The identified alternative fold is marked with an arrow. (H) X-ray crystal structure of zinc-free S4-1 polypeptide. The observed hydrogen bond more closely resembled the (i+3→i) 3₁₀ helix pattern observed in the original design than the pattern predicted in the alternative state. Nevertheless, the major shift, burying apolar side-chains, was observed. (I) Overlay of prediction and crystal structure for alternative apo state of S4-1 polypeptide. For clarity, only the backbone is shown. The RMSD between predicted alternative apo state and observed state in the crystal structure was 1 Å. (J) Ramachandran plots for the zinc-bound (top) and zinc-free (bottom) conformations. Curved arrows and numbers indicate progression through the sequence. Top panel: S4-symmetric designed zinc-bound conformation (two-toned points) and the observed zinc-bound conformation (solid colours matching panel B). All backbone dihedral values observed were very close to those designed. Bottom panel: predicted S4-symmetric alternative, zinc-free conformation and the observed symmetric structure (two-toned points). Although there was more deviation between the prediction and the observed structure, all points fell in the predicted Ramachandran bins. (K) Space-filling sphere models showing the major conformational change between zinc-bound (top) and zinc-free (bottom) states. Apolar side-chains that are solvent-exposed in the zinc-bound state are buried in the apo-state, while metal-binding histidine residues that are buried in the zinc-bound state are exposed in the apo-state, yielding a fully polar surface. (L) Cutaway views of zinc-bound (top) and zinc-free (bottom) S4-1 crystal structures. The zinc-bound state has a well-packed polar core consisting mainly of zinc-binding histidine side-chains (side-chains, top), with the central zinc atom lying on the symmetry axis and mirror plane. The apo-state has a well-packed apolar core more like that of a conventional protein (side-chains, bottom).

FIG. 4. Workflow for sampling quasi-symmetric conformations and designing symmetric peptides. (a) Overall workflow, showing progression from quasi-symmetric backbone conformational sampling through clustering, design, filtering, and fold propensity prediction. As one progresses through the pipeline, the number of models declines while the computational expenditure per model increases. (b) Method for sampling quasi-symmetric backbone conformations. The generalized kinematic closure (GenKIC) algorithm is used to sample closed loop conformations. Symmetric copies of degrees of freedom that can be randomized are randomized jointly. The solutions for the remaining degrees of freedom (which are solved for analytically in order to find closed macrocycle conformations) are filtered to discard solutions which break symmetry. This filtration involves a tolerance threshold, meaning that structures produced are nearly symmetric (quasi-symmetric), but not perfectly symmetric. (c) Pools of backbone conformations are clustered to reduce the number of redundant inputs into the design phase. For symmetric design, clustering was performed with cyclic permutation, where the permutation yielding the lowest RMSD to the cluster center was used to determine whether a given structure fell into the cluster. (d) Conversion of quasi-symmetric backbones to truly symmetric structures for symmetric sequence design. Quasi-symmetric macrocycles were aligned to the origin, with the Z-axis perpendicular to the plane of the macrocycle. All but one lobe were deleted, and the remaining lobe was copied with symmetric rotation (CN symmetry) or rotation with alternating inversion (SN symmetry). This operation left small backbone discontinuities which were corrected by symmetric, constrained energy minimization. RosettaDesign™ with symmetry was then carried out to find an energy-minimizing sequence for the given backbone conformation. (e) Designs were filtered to discard those with oversaturated hydrogen bond donors and acceptors (a known pathology favoured by the Rosetta™, energy function), as well as those with high total energy. (f) Designed sequences passing filters were assessed for propensity to favour the designed conformation (rigid folding) by large-scale sampling of alternative backbone conformations. Those designs with low-energy alternative states were discarded, and those with a unique low-energy state matching the design were accepted.

FIG. 5. Alternative crystal form of peptide S2-1. (a) Comparison between the conformation observed in this crystal form (left) and the designed conformation. Peptide S2-1 undergoes a major structural rearrangement compared to its other crystal form. The backbone heavy-atom RMSD between the observed conformation and the design was 3.3 Å. Note that this conformational change may have been facilitated by racemization at dSer1 (marked with an arrow), where both chiralities were observed in the electron density. (b) Racemic crystal packing observed for the alternative crystal form of peptide S2-1. Peptide S2-1 and its mirror image are shown. Calcium atoms facilitating intermolecular contacts are shown as spheres. (c) Crystal packing, shown along the needle axis. (d) A zippered pair of stacked sheets of peptide S2-1 and its mirror image (outlined with a dotted line in panel c). The peptide forms extensive intermolecular contacts, enabling the formation off a stacked p-sheet with peptides of the same chirality, and a leucine zipper between sheets of opposite chirality. Additionally, calcium atoms (spheres) bridge molecules. These extensive intermolecular interactions are likely strong enough to overcome the energetic disadvantage of the alternative conformation observed, facilitating conversion to this conformation.

### DETAILED DESCRIPTION

All references cited are herein incorporated by reference in their entirety. As used herein, the singular forms “a”. “an” and “the” include plural referents unless the context clearly dictates otherwise.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

Amino acid residues shown in upper case are L amino acids, and residues in lower case are D amino acids

In all embodiments of polypeptides disclosed herein, any N-terminal methionine residues are optional (i.e.: the N-terminal methionine residue may be present or may be absent).

All embodiments of any aspect of the disclosure can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

In one aspect, the disclosure provides polypeptides comprising or consisting of an amino acid sequence at least 66%, 70%, 75%, 80%, 82%, 84%, 86%, 88%, 90%, 91%, 92%, 93%, 94%, 95%, 96%, 97%, 98%, 99%, or 100% identical to the amino acid sequence selected from the group consisting of SEQ ID NO:1-91 (see Table 1), wherein:
(a) amino acid residues in upper case are L amino acids, and residues in lower case are D amino acids;
(b) X is 2-aminoisobutyric acid (AIB);
(c) no amino acid changes at proline or AIB residues in the reference peptide are permitted; and
(d) any amino acid changes must maintain chirality relative to the reference peptide (i.e.: L amino acids can be substituted by L amino acids only; D amino acids can be substituted by D amino acids only, and thus glycine and AIB (which are achiral) cannot be substituted into the polypeptide).

In one embodiment, the polypeptide contains 0, 1, or 2 amino acid changes relative to the reference peptide.

TABLE 1
C2 symmetric, 10 mer
seXSLseXSL (SEQ ID NO: 1) (C2-1), SEXslSEXsl (SEQ ID NO: 2)
ypDQSypDQS (SEQ ID NO: 3), YPdqsYPdqs (SEQ ID NO: 4)
DXRLqDXRLq (SEQ ID NO: 5), dXrlQdXrlQ (SEQ ID NO: 6)
PTeeyQPTeeyQ (SEQ ID NO: 7), ptEEYqptEEYq (SEQ ID NO: 8)
S2 symmetric, 8 mer
PeVkpEvK (SEQ ID NO: 9) (S2-1)
PkVepKvE (SEQ ID NO: 10) (S2-2)
S2 symmetric, 10 mer
qTRPDQtrpd (SEQ ID NO: 11) S2-3
EppKvePPkV (SEQ ID NO: 12) S2-4
pSEiRPseIr (SEQ ID NO: 13) S2-5
S2 symmetric, 12 mer
aNkhPeAnKHpE (SEQ ID NO: 14) S2-6
C3 symmetric, 9 mer
PRDPRDPRD (SEQ ID NO: 15) (C3-2), prdprdprd (SEQ ID NO: 16)
PDDPDDPDD (SEQ ID NO: 17) (C3-3), pddpddpdd (SEQ ID NO: 18)
C3 symmetric, 12 mer
PRVDPRVDPRVD (SEQ ID NO: 19) (C3-1), prVdprVdprVd (SEQ ID NO: 20)
C3 symmetric peptides
dlKdlKdlK (SEQ ID NO: 21); DLKDLKDLK (SEQ ID NO: 22)
DpYkDpYkDpYk (SEQ ID NO: 23), dPyKdPyKdPyK (SEQ ID NO: 24)
DpKyDpKyDpKy (SEQ ID NO: 25), dPkYdPKYdPKY (SEQ ID NO: 26)
DvpkDvpkDvpk (SEQ ID NO: 27), dVPKdVPKdVPK (SEQ ID NO: 28)
EPKEPKEPK (SEQ ID NO: 29), epkepkepk (SEQ ID NO: 30)
DPrYDPrYDPrY (SEQ ID NO: 31), dpRydpRydpRy (SEQ ID NO: 32)
EkAPEKAPEKAP (SEQ ID NO: 33), eKapeKapeKap (SEQ ID NO: 34)
KdpNLKdpNLKdpNL (SEQ ID NO: 35), kDPnlkDPnlkDPnl (SEQ ID NO: 36)
AvdQKAvdQKAvdQK (SEQ ID NO: 37), aVDqkaVDqkaVDqk (SEQ ID NO: 38)
dlqkPdlqkPdlqkP (SEQ ID NO: 39), DLQKpDLQKpDLQKp (SEQ ID NO: 40)
DlkDIKDIK (SEQ ID NO: 41), dLKdLKdLK (SEQ ID NO: 42)
pvrDpvrDpvrD (SEQ ID NO: 43), PVRdPVRdPVRd (SEQ ID NO: 44)
RePIRePIRePI (SEQ ID NO:45), rEpirEpirEpi (SEQ ID NO: 46)
KPVDKPVDKPVD (SEQ ID NO: 47), kpvdkpvdkpvd (SEQ ID NO: 48)
DKTLDKTLDKTL (SEQ ID NO: 49), dktldktldktl (SEQ ID NO: 50)
dPyRdPyRdPyR (SEQ ID NO: 51), DpYrDpYrDpYr (SEQ ID NO: 52)
DKVYDKVYDKVY (SEQ ID NO: 53), dkVydkVydkVy (SEQ ID NO: 54)
DIRSDIRsDIRs (SEQ ID NO: 55); dLrSdLrSdLrS (SEQ ID NO: 56)
PeRPeRPeR (SEQ ID NO: 57), pErpErpEr (SEQ ID NO: 58)
PQKDVPQKDVPQKDV (SEQ ID NO: 59), pqKdVpqKdVpqKdV (SEQ ID NO: 60)
lpEASKlpEASKlpEASK (SEQ ID NO: 61), LPeaskLPeaskLPeask (SEQ ID
NO: 62)
iKepiKepiKep (SEQ ID NO: 63), IKEPIKEPIKEP (SEQ ID NO: 64)
PTKDvPTKDvPTKDV (SEQ ID NO: 65), ptkdVptkdVptkdV (SEQ ID NO: 66)
PEIPEIPEI (SEQ ID NO: 67), peipeipei (SEQ ID NO: 68)
PHIPHIPHI (SEQ ID NO: 69), phiphiphi (SEQ ID NO: 70)
PNDPNDPND (SEQ ID NO: 71), pndpndpnd (SEQ ID NO: 72)
C4 symmetric peptides
dXyKdXyKdXyKdXyK (SEQ ID NO: 73), DXYkDXYKDXYKDXYK (SEQ ID NO: 74)
etPKetPKetPKetPK (SEQ ID NO: 75), ETpkETpkETpkETpk (SEQ ID NO: 76)
CS symmetric peptides
PKDPKDPKDPKDPKD (SEQ ID NO: 77), pkdpkdpkdpkdpkd (SEQ ID NO: 78)
pRdpRdpRdpRdpRd (SEQ ID NO: 79), PrDPrDPrDPrDPID (SEQ ID NO: 80)
HKDHKDHKDHKDHKD (SEQ ID NO: 81), hkdhkdhkdhkdhkd (SEQ ID NO: 82)
dlRdlRdlRdlRdIR (SEQ ID NO: 83), DLrDLrDLrDLrDLr (SEQ ID NO: 84)
vRdvRdvRdvRdvRd (SEQ ID NO: 85), VrDVEDVIDVEDVID (SEQ ID NO: 86)
dKeTdKeTdKeTdKeTdKeT (SEQ ID NO: 87), DKEtDkEtDkEtDkEtDkEt
(SEQ ID NO: 88)
FTApFTApFTApFTApFTAp (SEQ ID NO: 89), ftaPftaPftaPftaPftaP
(SEQ ID NO: 90)
S4 symmetric 24mer
KLqeXHklQEXhKLqeXHklQEXh (SEQ ID NO: 91) (S4-1)

The inventors have shown that the polypeptides of the disclosure are mixed-chirality peptide macrocycles with rigid structures that feature internal cyclic symmetries or improper rotational symmetries inaccessible to natural proteins, and can be used, for example, in therapeutic and nanomaterial design, as well as in synthetic switching systems and in co-assemblies with metals to form super molecular crystals.

The polypeptides are cyclic peptides in that there is a covalent linkage between the residues shown as N-terminal and C-terminal in SEQ ID NOS:1-91. Each of the polypeptides includes a series of repeats. For example:
seXSLseXSL (SEQ ID NO:1) includes two repeats of the peptide “seXSL”;
PkVepKvE (SEQ ID NO:10) includes two repeats of the residues “PkVe”, though the second repeat has opposite chirality (pKvE);
PRDPRDPRD (SEQ ID NO:15) includes three repeats of the peptide “PRD”;
dXvKdXvKdXyKdXyK (SEQ ID NO:73) includes 4 repeats of the peptide “dXyK”;
pRdpRdpRdpRdpRd (SEQ ID NO:79) includes 5 repeats of the peptide “pRd”; and
KLqeXHklQEXhKLqeXHkIQEXh (SEQ ID NO:91) includes 4 repeats of the peptide “KLqeXH”, though in alternating chirality (i.e.: the 1^{st} and 3^{rd} repeats are KLqcXH, and the 2^{nd} and 4^{th} repeats are “klQEXh”)

In one embodiment wherein the polypeptides are mutated relative to the reference polypeptide, the same mutation (i.e.: the same amino acid residue substitution) is made to each repeat unit. In another embodiment, no proline or AIB residues may be added by amino acid change relative to the reference polypeptide.

In one embodiment, the polypeptide has C2 symmetry, and comprises an amino acid sequence at least 66%, 70%, 75%, 80%, 82%, 84%, 86%, 88%, 90%, 91%, 92%, 93%, 94%, 95%, 96%, 97%, 98%, 99%, or 100% identical to the amino acid sequence selected from the group consisting of SEQ ID NO:1-8.

In another embodiment, the polypeptide has S2 symmetry, and comprises an amino acid sequence at least 66%, 70%, 75%, 80%, 82%, 84%, 86%, 88%, 90%, 91%, 92%, 93%, 94%, 95%, 96%, 97%, 98%, 99%, or 100% identical to the amino acid sequence selected from the group consisting of SEQ ID NO:9-14.

In a further embodiment, the polypeptide has C3 symmetry, and comprises an amino acid sequence at least 66%, 70%, 75%, 80%, 82%, 84%, 86%, 88%, 90%, 91%, 92%, 93%, 94%, 95%, 96%, 97%, 98%, 99%, or 100% identical to the amino acid sequence selected from the group consisting of SEQ ID NO: 15-72. In one such embodiment of the C3 symmetry polypeptides, the first residue of the asymmetric repeat unit is L-Proline and the third residue of the asymmetric unit is L-Aspartic acid, and the 2nd residue can be any non-glycine, non-proline, non-AIB L-amino acid

In one embodiment, the polypeptide has C4 symmetry, and comprises an amino acid sequence at least 66%, 70%, 75%, 80%, 82%, 84%, 86%, 88%, 90%, 91%, 92%, 93%, 94%, 95%, 96%, 97%, 98%, 99%, or 100% identical to the amino acid sequence selected from the group consisting of SEQ ID NO:73-76. In one such embodiment, the polypeptide is bound to a metal ion, including but not limited to Zn2+. In this embodiment, the polypeptide may undergo a conformational change when bound to the metal ion as opposed to the unbound state, and thus may be used, for example, in detection of metal ions, or as a synthetic switching systems and in co-assemblies with metals to form super molecular crystals.

In another embodiment, the polypeptide has C5 symmetry, and comprises an amino acid sequence at least 66%, 70%, 75%, 80%, 82%, 84%, 86%, 88%, 90%, 91%, 92%, 93%, 94%, 95%, 96%, 97%, 98%, 99%, or 100% identical to the amino acid sequence selected from the group consisting of SEQ ID NO:77-90.

In a further embodiment, the polypeptide has S4 symmetry, and comprises an amino acid sequence at least 66%, 70%, 75%, 80%, 82%, 84%, 86%, 88%, 90%, 91%, 92%, 93%, 94%, 95%, 96%, 97%, 98%, 99%, or 100% identical to the amino acid sequence of SEQ ID NO:91.

In all of these embodiments, the polypeptide may be conjugated to one or more additional components. In one embodiment, the one or more additional components may be cross-linked to a side chain of an amino acid residue in the polypeptide using conventional techniques. Any additional component may be conjugated to the polypeptides of the disclosure as appropriate for an intended use, including but not limited to detectable tags, small molecules, radioactive agents, antibodies, polyethylene glycol, therapeutic moieties, and diagnostic moieties. In all of these embodiments, the percent identity requirement does not include any additional functional domain that may be conjugated to the polypeptide.

In another embodiment, the disclosure provides compositions, comprising a plurality of the polypeptides of the disclosure attached to a scaffold. Any suitable scaffold may be used, including but not limited to polypeptide scaffolds, beads, virus-like particles, etc.

The polypeptides of the disclosure may be chemically synthesized using any suitable technique. Those polypeptides of the disclosure that include only L amino acids (SEQ ID NOs: 15, 17, 29, 47, 49, 67, 69, 71, 77, and 81) may be expressed recombinantly.

The disclosure also provides nucleic acids encoding a polypeptide comprising or consisting of an amino acid sequence at least 66%, 70%, 75%, 80%, 82%, 84%, 86%, 88%, 90%, 91%, 92%, 93%, 94%, 95%, 96%, 97%, 98%, 99%, or 100% identical to the amino acid sequence selected from the group consisting of SEQ ID NO: 15, 17, 29, 47, 49, 67, 69, 71, 77, and 81, wherein:
(a) amino acid residues in upper case are L amino acids;
(b) no amino acid changes at proline residues in the reference peptide are permitted; and
(d) any amino acid changes must maintain chirality relative to the reference peptide.

The nucleic acid sequence may comprise single stranded or double stranded RNA or DNA in genomic or cDNA form, or DNA-RNA hybrids, each of which may include chemically or biochemically modified, non-natural, or derivatized nucleotide bases. Such nucleic acid sequences may comprise additional sequences useful for promoting expression and/or purification of the encoded protein, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the proteins of the disclosure.

In a further aspect, the disclosure provides expression vectors comprising nucleic acids of the disclosure operatively linked to a control sequence. “Expression vector” includes vectors that operatively link a nucleic acid coding region or gene to any control sequences capable of effecting expression of the gene product. “Control sequences” operatively linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type, including but not limited plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The expression vector must be replicable in the host organisms either as an episome or by integration into host chromosomal DNA. In various embodiments, the expression vector may comprise a plasmid, viral-based vector, or any other suitable expression vector.

In one aspect, the disclosure provides recombinant host cell comprising the proteins, nucleic acids and/or the expression vectors of any embodiment or combination of embodiments of the disclosure. The host cells can be either prokaryotic or eukaryotic. The cells can be transiently or stably engineered to incorporate the expression vector of the invention, using techniques including but not limited to bacterial transformations, calcium phosphate co-precipitation, electroporation, or liposome mediated-, DEAE dextran mediated-, polycationic mediated-, or viral mediated transfection. (See, for example, Molecular Cloning: A Laboratory Manual (Sambrook, et al., 1989, Cold Spring Harbor Laboratory Press); Culture of Animal Cells: A Manual of Basic Technique, 2^{nd} Ed. (R. I. Freshney. 1987. Liss, Inc. New York, N.Y.)). A method of producing a protein according to the invention is an additional part of the invention. The method comprises the steps of (a) culturing a host according to this aspect of the invention under conditions conducive to the expression of the protein, and (b) optionally, recovering the expressed protein. The expressed protein can be recovered from the cell free extract, but preferably they are recovered from the culture medium.

In another aspect, the disclosure provides methods for use of the polypeptides of any embodiment or combination of embodiments herein for any suitable use, including but not limited to therapeutic and nanomaterial design, biosensors, as well as in synthetic switching systems and in co-assemblies with metals to form super molecular crystals.

In a further aspect, the disclosure provides method for designing mixed chirality peptide macrocycles with internal symmetry according to any embodiment or combination of embodiments described herein. Exemplary such methods are detailed in the examples that follow.

### Examples

Cyclic symmetry is frequent in protein and peptide homo-oligomers, but extremely rare within a single chain, as it is not compatible with free N and C termini. Here we describe the computational design of mixed-chirality peptide macrocycles with rigid structures that feature internal cyclic symmetries or improper rotational symmetries inaccessible to natural proteins. Crystal structures of three C2- and C3-symmetric macrocycles, and of six diverse S2-symmetric macrocycles, match the computationally-designed models with backbone heavy-atom RMSD values of 1 Å or better. Crystal structures of an S4-symmetric macrocycle (consisting of a sequence and structure segment mirrored at each of three successive repeats) designed to bind zinc reveal a large-scale zinc-driven conformational change from an S4-symmetric apo-state to a nearly inverted S4-symmetric holo-state almost identical to the design model. This work demonstrates the power of computational design for exploring symmetries and structures not found in nature, and for creating synthetic switchable systems.

We set out to develop general methods for computationally designing internally-symmetric peptide macrocycles with conformational rigidity imparted by large energy gaps between a symmetric ground state and all alternative conformations. To this end, we incorporated methods for sampling and designing with internal cyclic or improper rotational symmetries into the Rosetta™ heteropolymer design software. Our sampling methods use kinematic closure methods to provide analytical solutions for dihedral values yielding closed macrocycle conformations. Dihedral angles in all asymmetric units of the macrocycle (henceforth referred to as “lobes”) are required to match those in the first “reference” lobe to within a certain tolerance in the cyclic symmetric case, and after inversion in the case of improper rotational symmetries. Subsequent symmetric sequence design algorithms ensure residue identities and conformations in neighboring lobes match (for cyclic symmetry) or match with chirality inversion and inversion of dihedral values (for improper rotational symmetry).

Here, we apply the newly-developed computational methods to the creation of peptide macrocycles with cyclic symmetries (C2 or C3). We also explore the structures possible with improper rotational symmetries that are inaccessible to homochiral proteins, but which can be accessed by heterochiral peptides, demonstrating robust ability to design diverse, internally S2-symmetric folds. Finally, we present an S4-symmetric polypeptide macrocycle that functions as a conformational switch, inverting its fold in the presence or absence of zinc. Exemplary polypeptide designs are shown in Table 1. The robust computational design and validation methods demonstrated here are applicable to diverse problems in therapeutic and nanomaterial design.

Structures of Designed Peptide Macrocycles with C2 and C3 Symmetry

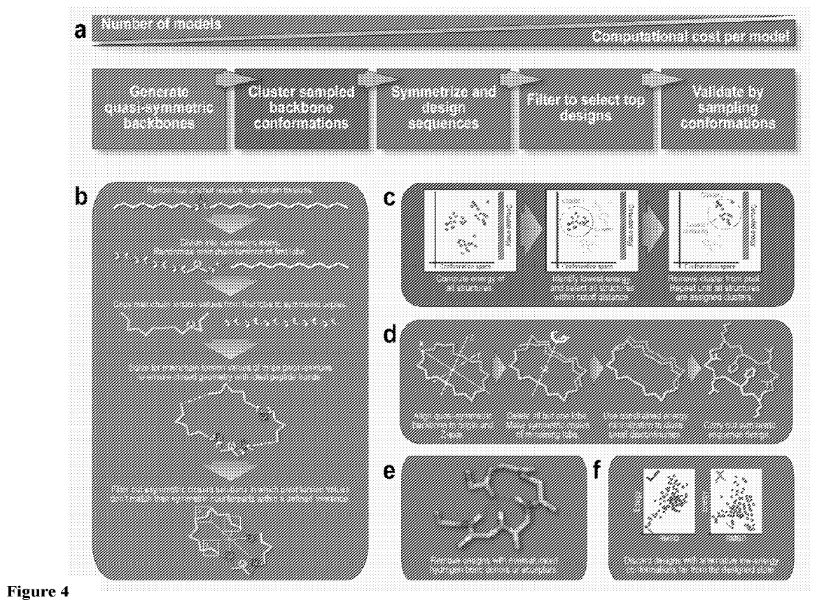

We developed a computational pipeline, summarized in the methods and in FIG. 4 in the supplementary information, for designing internally-symmetric peptide macrocycles. This pipeline involved steps of symmetric backbone conformational sampling, clustering, symmetric sequence design, filtration to discard designs with undesirable features, and final computational validation by large-scale conformational sampling. We first applied this pipeline to create macrocycles with C2 and C3 symmetry, with asymmetric units ranging from 3 to 5 residues. To assess the completeness of our sampling of cyclic peptide conformational space, we defined four backbone dihedral bins (A, B, X, and Y), with A and B representing right-handed helical and strand regions of Ramachandran space, respectively, and x and y representing the mirror image bins. We compared the number of bin sequences (AABAYYXY, for example) sampled to the total number of unique bin patterns possible for each peptide size and symmetry type designed, with the latter determined analytically using Burnside's lemma (Burnside, 1900). Where possible, we also assessed whether the solution space for designs was fully sampled by examining the set of low-energy structures obtained to determine whether both members of mirror-related bin strings were represented, since the occurrence of just one member of such isoenergetic pairs indicates incomplete sampling of the solution space. We were able to sample with many-fold redundancy over all bin patterns possible for peptides up to 24 residues in length, and found only a small subset for each length that were compatible with chain closure for each symmetry (Table 2). The mirror test suggested that the identified conformations completely cover the space of possibilities for C2-symmetric macrocycles with up to 8 residues, and C3-symmetric macrocycles up to 18 residues. For higher-order symmetries, such as C5, complete sampling by the mirror test was possible up to 30 residues (data not shown).

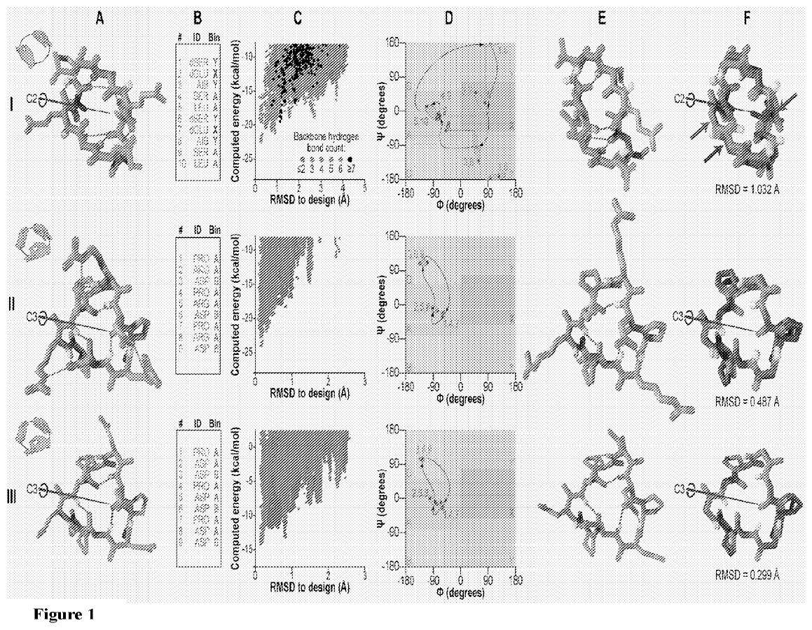

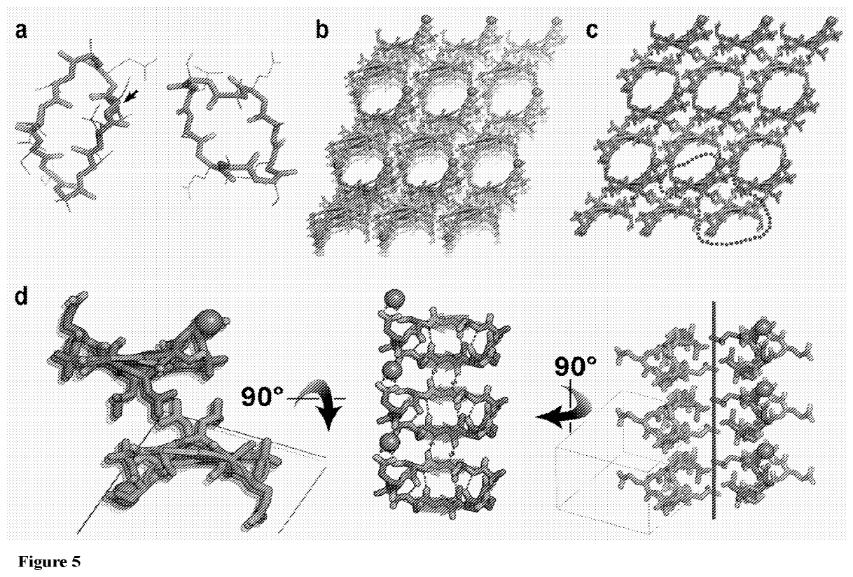

We carried out large-scale conformational sampling on each of the designed sequences, and selected peptide macrocycles with large energy gaps between the designed conformation and all alternative states to synthesize chemically. We began by synthesizing one C2-symmetric and five C3-symmetric peptide macrocycles and their mirror-image enantiomorphs to facilitate the crystallization of the synthesized molecules from racemic mixture (Yeates and Kent, 2012). With this approach we succeeded in crystallizing the C2-symmetric peptide and three of the five C3-symmetric peptides (FIG. 1). The observed conformation of the C2 symmetric peptide (designated C2-1) matched its designed conformation within a backbone heavy-atom RMSD of 1 Å, but with notable deviation in the W dihedral angle of residue dSER1 and the dihedral angles of residues dGLU2 and A1B8, which inverted the dSER1-dGLU2 amide bond, allowing it to form a new hydrogen bond with dGLU7. Despite this local structural change, the overall fold of the peptide was largely preserved. This is the first computationally-designed macrocycle of which we are aware that uses 2-aminoisobutyric acid (AIB), a non-canonical, conformationally-constrained amino acid, instead of proline to ensure conformational rigidity. Only one enantiomer was observed in this crystal structure, but an alternative crystal form was identified that incorporates both enantiomers, albeit with a distorted conformation (see FIG. 5, supplementary methods).

Two of the three C3-symmetric peptides that we succeeded in crystallizing closely matched the design models (FIG. 1). Design C3-adopted a conformation different from the design, likely due to three buried polar hydrogen atoms that form hydrogen bonds with carbonyl oxygens in adjacent peptide molecules (Ghadiri et al., 1993; Ranganathan et al., 2000). Recognizing this to be an undesirable structural feature that was poorly penalized by our automated scoring function, from this point forward we filtered designs, selecting only those designs lacking unsatisfied buried polar groups. Crystal structures of peptides C3-2 and C3-3, which lack buried unsatisfied polar atoms, closely matched their designs, with backbone heavy-atom RMSD values of 0.5 Å and 0.3 Å, respectively. The two have a common fold stabilized by three proline residues and three backbone hydrogen bonds.

Structures of Designed Peptide Macrocycles with S2 Symmetry

We next explored symmetries inaccessible to natural proteins. Unlike proteins built only from L-amino acids, peptides built from mixtures of D- and L-amino acids can access symmetries involving mirror operations, such as improper rotational symmetries. Using the same symmetric sampling, clustering, and sequence optimization strategy, we designed and synthesized a panel of 6 peptide macrocycles with S2 symmetry ranging in size from 8 to 12 amino acids (designs S2-1 through S2-6). These peptides have a sequence that repeats twice, with the chirality of residues in the second lobe inverted relative to the first, yielding a second lobe with a conformation mirroring that of the first. Six designs were selected for synthesis representing a diverse range of backbone conformations and hydrogen bonding patterns. We were able to crystallize all six of these peptides, and to determine their structures by direct phasing methods.

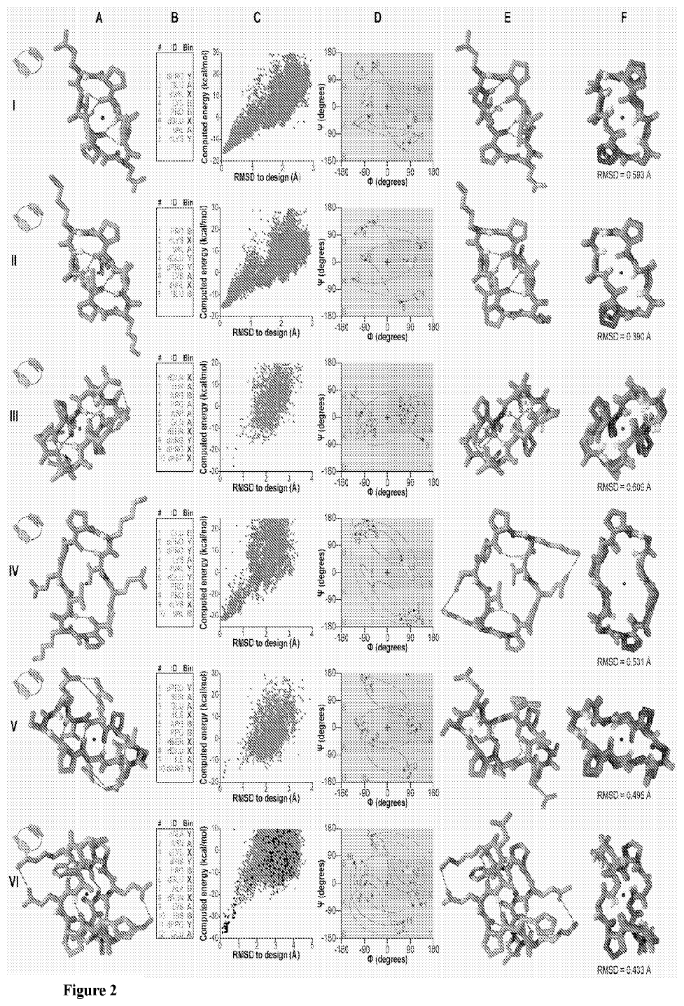

In all cases the observed conformation closely matched the design (overlays in FIG. 2, column F), with a maximum backbone heavy-atom RMSD of 0.6 Å (peptide S2-3) and a minimum of 0.4 Å (peptide 52-1). These peptides' folds were all stabilized by D- and L-proline residues and by backbone hydrogen bonds (FIG. 2). The designed conformation of peptide S2-6, a 12-residue peptide with sequence aNkhPeAnKHpE (SEQ ID NO:14) (where lowercase and uppercase letters represent D- and L-amino acid residues, respectively), has a crimped conformation remarkably forming 8 backbone hydrogen bonds, of which 6 were preserved in the crystal structure. The remaining two were lost to a slight rotation of the amide bond between dASN8 and LYS9, which positioned the donor and acceptor groups where they could instead make hydrogen bonds to water. The crystal structure deviated from the design by a backbone heavy-atom RMSD of only 0.4 Å. In all other cases, the designed backbone hydrogen bonding patterns were preserved, with two exceptions: a subtle relaxation of the backbones of peptides S2-4 and S2-5 replaced two direct backbone-backbone hydrogen bonds in the designs with bridging water molecules (FIGS. 2d and e).

### Metal-Induced Conformational Switching in a Designed S4-Symmetric Polypeptide Macrocycle

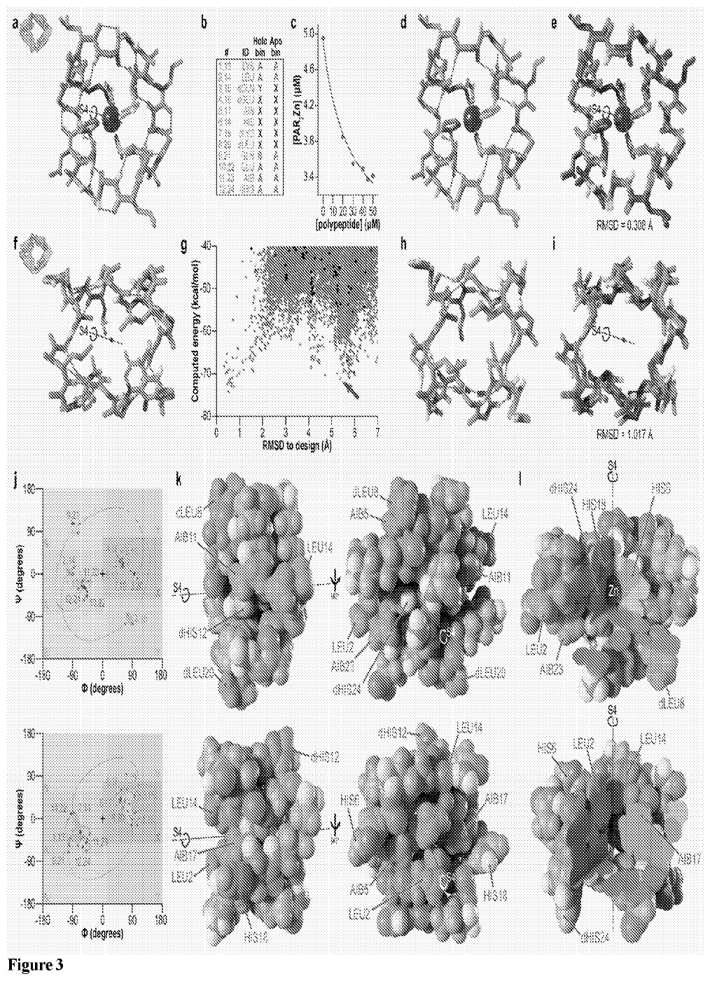

We next explored the possibility of using the new design methods to create conformational switches. We sampled S4-symmetric polypeptide conformations with 4 repeats in which alternating lobes had opposite chirality, and developed a computational strategy to select backbones that could present metal-binding side-chains for tetrahedral coordination of a central metal ion (see supplementary information). We designed sequences with L- and D-histidine residues positioned to chelate the metal ion, AIB and other residues to stabilize the fold, and apolar side-chains on the surface to stabilize an alternative, inside-out conformation in the absence of metal. The sequences of these designs repeat four times, with residues in the second and fourth lobes possessing chirality and conformations inverted relative to equivalent residues in the first and third lobes. We carried out large-scale conformational sampling to select designs with low-energy designed conformations with the histidine side-chains positioned to bind zinc. In several designs, we noted that large-scale conformational sampling predicted a second energy minimum corresponding to an inside-out fold with the apolar side-chains in the core and the histidines exposed. We selected a single design for synthesis, S4-1, which has sequence KLgeXHklQEXhKLqeXHklQEXh (SEQ ID NO:91), in which x represents AIB (FIG. 3).

The 24-amino acid polypeptide S4-1 crystallized in space group P1̅. The backbone conformation in the crystal structure (which contained a single copy of the peptide in the asymmetric unit), matched the design with a backbone heavy-atom RMSD of 0.3 Å, with a central metal ion (believed to be zinc) very close to that in the design model. The central metal-coordinating L- and D-histidine side-chains matched the design with a side-chain heavy-atom RMSD of 0.1 Å. The zinc affinity of S4-1 was 0.32 nM, as measured by competition with the colorimetric chelator 4-(2-pyridylazo)resorcinol (PAR) (FIG. 3C and supplementary methods).

The central metal ion plays an important structural role in holo-S4-1, stabilizing a conformation that presents four apolar D- and L-leucine side-chains to aqueous solvent (FIGS. 3A, D-E, K-L), and as noted above large-scale conformational sampling runs predicted an alternative conformation with these groups buried in the absence of metal ion (FIGS. 3F and G). To explore this possibility, we solved the structure of the apo-polypeptide. Without zinc, the polypeptide crystallized in space group P2₁2₁2, and did indeed adopt a very different conformation from the holo-structure, packing apolar D- and L-leucine side-chains against AIB residues in the core, and projecting the D- and L-histidine side-chains that previously coordinated zinc outward (FIGS. 3H, K-L). The observed apo-state conformation matches the predicted alternative state to a backbone heavy-atom RMSD of 1 Å (FIG. 31).

### Discussion

The new peptide and polypeptide macrocycles presented here have diverse, rigid backbone folds closely matching the design models, in nearly all cases with sub-Angstrom accuracy. The structures were designed in four different symmetry classes (C2, C3, S2, and S4), the latter two of which are inaccessible to proteins or other natural macromolecules built from building-blocks of only one handedness. Likely because of their symmetry, the success rate of the designs was quite high, both in terms of crystallization of the designed peptides (11 of 13) and their close match to predicted structures (10 of 11). Since synthetic macrocycles are not limited to the 20 canonical amino acid building-blocks, we take advantage of the non-canonical, conformationally-constrained amino acid residue AIB to rigidify two macrocycles. We also illustrate the use of metal ligands as central structural elements. Moving forward the methods described here can be used to design with the thousands of possible non-canonical amino acids, as well as with bound metal ions.

### Near-Exhaustive Exploration of Conformational Space for Larger Molecules

The design of rigidly-folded heteropolymers requires efficient means of sampling backbone conformations, both to identify conformational states compatible with a given function that can be stabilized by a suitable choice of sequence, and to validate designed sequences by exploring possible alternative low-energy conformational states. This is particularly challenging when designing with non-canonical amino acid residues, requiring unbiased sampling methods that are not reliant on known structures. Because N-fold symmetric molecules have far fewer (1/N) conformational degrees of freedom than similarly sized asymmetric molecules, the new methods make comprehensive sampling tractable for much larger systems. By focusing on internally-symmetric macrocycles, we were able to achieve exhaustive or near-exhaustive coverage of the conformation spaces for peptides with up to 30 amino acids for the highest-order symmetries, a size range well beyond that which can be sampled exhaustively for asymmetric macrocycles. Since many applications of designed, well-folded heteropolymers require molecules that are able to present large binding interfaces (e.g. for nanomaterial self-assembly or therapeutic target binding), or molecules large enough to possess internal binding pockets (e.g. for small-molecule binding or catalysis), we anticipate that our computational methods for designing larger symmetric structures with non-canonical building blocks will have broad applicability.

### Design of Metal-Dependent Peptide Conformational Dynamics

Our most complex design, the 24-residue S4-1 polypeptide, binds zinc with sub-nanomolar affinity, and undergoes a major conformational change when zinc is removed. Both the apo and holo states are well-structured, with the former burying apolar side-chains and the latter exposing them (FIG. 3). Engineered switching behaviour could ultimately be used to create cell-permeable molecules that could serve as drugs. The ability to control this switching behaviour with a metal ion like zinc could also provide a means of ensuring that the permeation is unidirectional: while extracellular free zinc concentrations are in the nanomolar to micromolar range (Frederickson et al., 2006), high-affinity zinc binding within the cell lowers the free zinc concentration to picomolar levels (Maret, 2017).

### Concluding Remarks

We have presented general methods for computational design and validation of symmetric, well-folded polypeptide macrocycles, including those incorporating metals as structural elements, and have demonstrated robust ability to control structure with sub-Angstrom accuracy, culminating in an engineered, metal-dependent conformational switch. The ability to design symmetric, well-folded polypeptide macrocycles opens up new avenues for both therapeutic design and for bounded and unbounded nanomaterial design, and shows that methods originally developed for protein design can now be used to robustly design molecules quite unlike those that exist in nature.

### Materials and Methods:

### Modifications to the Rosetta™ Software Suite

Extensive modifications to the Rosetta™ software suite enabled the design of internally-symmetric peptide macrocycles, including those able to coordinate a central metal ion. New Rosetta™ modules were implemented to be compatible with both the PyRosetta™ and RosettaScripts™ scripting languages (Chaudhury et al., 2010; Fleishman et al., 2011), allowing their use in the development of future, application-specific design protocols.

The Rosetta™ symmetry code (DiMaio et al., 2011) was refactored to add support for mirror operations and improper rotational symmetries, and to correctly interconvert between mirrored amino acid types. Rosetta™ simple_cycpep_predict and energy_based_clustering applications, both described previously (Bhardwaj et al., 2016; Hosseinzadeh et al., 2017), were enhanced to allow sampling and clustering of quasi-symmetric backbones with a given symmetry (where a quasi-symmetric backbones is one that is nearly symmetric, but in which small deviations from perfect symmetry are allowed). A Rosetta™ module (“mover”) for converting quasi-symmetric structures to fully symmetric structures, called the SymetriCycpepAlignMover, was added. The interface and internal handling of non-canonical amino acids during design was greatly reworked, with the user-controlled PackerPalette introduced to control the set of chemical building-blocks used for a given design task, permitting deprecation of many problematic idiosyncrasies present in the previous interface to streamline the design process.

To enable the design of metal-binding peptides, Rosetta™ CrosslinkerMover was enhanced with support for a range of metal coordination geometries, with support for asymmetric structures or for the symmetry classes compatible with a given coordination geometry. For example, this mover allows the design of a tetrahedrally-coordinated zinc in an asymmetric structure or in a C2 or S4-symmetric structure, with suitable repetition of conformations and amino acid identities of the liganding residues.

### Computational Design Protocol

To design symmetric peptides, we first sampled quasi-symmetric mainchain conformations using the simple_cycpep_predict application, and enumerated conformations with the energy_based_clustering application. In the case of the S4-1 polypeptide, this step was modified to sample only those backbone conformations capable of coordinating a central metal ion. Next, with scripts written in the RosettaScripts™ scripting language, we converted quasi-symmetric cluster centers to fully symmetric structures, and carried out sequence design with Rosetta™ symmetric design algorithms. Finally, we validated each designed sequence by large-scale conformational sampling, again using the simple_cypep_predict application, to identify those designed sequences that uniquely favoured the designed conformation. Computations were carried out on the University of Washington Hyak cluster, the Argonne National Laboratory Mira and Theta supercomputers, and the Simons Foundation Gordon and Iron clusters. Additionally, some validations were carried out using the Rosetta@Home™ distributed computing platform, which uses volunteer computers, cellular telephones, and mobile devices through the Berkeley Open Infrastructure for Network Computing (BOINC) (Anderson, 2004).

### Solid-Phase Peptide Synthesis and Purification

Peptides were synthesized using standard Fmoc solid-phase peptide synthesis techniques using a CEM Liberty Blue peptide synthesizer with microwave-heated coupling and deprotection steps. Peptides with twelve amino acids or fewer that contained L-aspartate or L-glutamate were synthesized tethered by the acidic side-chain to preloaded Fmoc-L-Asp(Wang resin LL)-ODmab or Fmoc-L-Glu(Wang resin LL)-ODmab resin, and were cyclized on-bead by a coupling reaction following deprotection of the C-terminus with 2% (v/v) hydrazine monohydrate treatment in dimethylformamide (DMF). Larger peptides were synthesized with the C-terminus coupled to Cl-TCP(Cl) resin from CEM, cleaved from the resin with 1% (v/v) TFA treatment in dicholoromethane (DCM), and cyclized by a solution-phase coupling reaction prior to final deprotection. Peptides were purified by reverse-phase HPLC with a water-acetonitrile gradient, lyophilized, and redissolved in buffer suitable for subsequent experiments (typically 100 mM HEPES, pH 7.5). Masses and purities were assessed by electrospray ionization mass spectrometry with a Thermo Scientific TSQ Quantum Access™ mass spectrometer. Full synthetic and purification protocols are described in the supplementary information.

### X-Ray Crystallography

Peptides were crystallized by hanging droplet vapour diffusion, with pH, buffer, ionic strength, and precipitants all optimized for each peptide. Growth conditions for the crystals of each peptide are described in the supplementary information. Diffraction data were collected at the Argonne National Laboratory Advanced Photon Source (APS) beamlines 24-ID-C and 24-ID-E.

### Metal-Binding Assays

To confirm zinc content of the 4-1 and 4-2 peptides, and to measure zinc affinity, we used a variant of the 4-(2-pyridylazo)resorcinol (PAR) assay described previously (Crow et al., 1997; Hunt et al., 1985, Mulligan et al., 2008). We carried this assay out in 96-well plates (200 μl total solution volume per well). To confirm metal content, 10 μM polypeptide was denatured in 6 M guanidinium hydrochloride (Sigma-Aldrich, St. Louis, Mo.), 100 mM HEPES, pH 7.5, and 200 μM PAR, and the change in absorbance at 490 nm was monitored using a SpectraMAX™ M5e plate reader (Molecular Devices, San Jose, Calif.). Standard curves were prepared with ZnCl₂ to convert absorbance changes to zinc concentrations. The metal affinity of the S4-1 and S4-2 polypeptides was measured by competition with PAR, given the known dissociation constants of the PAR₂-Zn complex. Full protocols and mathematical details for both assays are provided in the supplementary information.

### Data Tables

TABLE 2
Summary of peptide macrocycle backbone conformations
found for symmetries explored.

| Symmetry | Size | Max. bin strings | Bin strings | Conformational |
|---|---|---|---|---|
| type¹ | (residues) | possible | observed | clusters observed² |
| C2 | 6 | 24 | 8 | 3 |
| | 8 | 70 | 13 | 6 |
| | 10 | 208 | 57 | 25 |
| | 12 | 700 | 15 | 16 |
| | 14 | 2,344 | 108 | 96 |
| | 16 | 8,230 | 1,122 | 898 |
| | 18 | 29,144 | 2,171 | 1,770 |
| | 20 | 104,968 | 2,585 | 2,108 |
| C3 | 6 | 10 | 1 | 1 |
| | 9 | 24 | 4 | 2 |
| | 12 | 70 | 3 | 3 |
| | 15 | 208 | 4 | 4 |
| | 18 | 700 | 3 | 3 |
| | 21 | 2,344 | 14 | 11 |
| | 24 | 8,230 | 20 | 19 |
| S2 | 6 | 12 | 1 | 1 |
| | 8 | 32 | 2 | 1 |
| | 10 | 104 | 9 | 6 |
| | 12 | 344 | 4 | 4 |
| | 14 | 1,172 | 173 | 85 |
| | 16 | 4,096 | 294 | 162 |
| | 18 | 14,572 | 469 | 303 |
| | 20 | 52,432 | 707 | 464 |
| S4 | 8 | 4 | 2 | 1 |
| | 12 | 12 | 1 | 1 |
| | 16 | 32 | 7 | 7 |
| | 20 | 104 | 22 | 19 |
| | 24 | 344 | 18 | 17 |
| | 28 | 1,172 | 27 | 22 |
| | 32 | 4,096 | 13 | 12 |

¹Only symmetry types that were synthesized are listed here. For full analysis of other symmetry types (e.g. C4, C5), please see the supplementary information.
²Results of clustering with a radius of 1.5 Å are reported. For full clustering analysis, please refer to the supplementary information.

### REFERENCES

Anderson D P. 2004. BOINC: A System for Public-Resource Computing and Storage Fifth IEEE/ACM International Workshop on Grid Computing. Presented at the Fifth IEEE/ACM International Workshop on Grid Computing. Pittsburgh, Pa., USA: IEEE. pp. 4-10.doi:10.1109/GRID.2004.14
Bennett W M, Norman D J. 1986. Action and toxicity of cyclosporine. Annu Rev Med 37:215-224. doi:10.1146/annurev.mc.37.020186.001243
Bhardwaj G, Mulligan V K, Bahl C D, Gilmore J M, Harvey P J, Cheneval O, Buchko G W, Pulavarti SVSRK, Kaas Q, Eletsky A, Huang P-S, Johnsen W A, Greisen P J, Rocklin G J, Song Y, Linsky I V, Watkins A, Rettie S A, Xu X, Carter L P, Bonneau R I, Olson J M, Coutsias E, Correnti C E, Szyvperski T, Craik D J, Baker D. 2016. Accurate de novo design of hyperstable constrained peptides. Nature 538:329-335.
Burnside W. 1900. On Some Properties of Groups of Odd Order. Proceedings of the London Mathematical Society 33:162-184.
Chaudhury S, Lyskov S, Gray J J. 2010. PyRosetta: a script-based interface for implementing molecular modeling algorithms using Rosetta. Bioinformatics 26:689-691. doi:10.1093/bioinformatics/btq007
Conti E, Stachelhaus T, Marahiel M A, Brick P. 1997. Structural basis for the activation of phenylalanine in the non-ribosomal biosynthesis of gramicidin S. EMBO J 16:4174-4183. doi:10.1093/emboj/16.14.4174
Coutsias E A, Seok C, Jacobson M P, Dill K A. 2004. A kinematic view of loop closure. J Comput Chem 25:510-528. doi:10.1002/jcc.10416
Crow J P, Sampson J B, Zhuang Y, Thompson J A, Beckman J S. 1997. Decreased zinc affinity of amyotrophic lateral sclerosis-associated superoxide dismutase mutants leads to enhanced catalysis of tyrosine nitration by peroxynitrite. J Neurochem 69:1936-1944. doi:10.1046/j.1471-4159.1997.69051936.x
Dang B, Wu H, Mulligan V K, Mravic M, Wu Y, Lemmin T, Ford A, Silva D-A, Baker D, DeGrado W F. 2017. De novo design of covalently constrained mesosize protein scaffolds with unique tertiary structures. Proceedings of the National Academy of Sciences 114:10852-10857. doi:10.1073/pnas.1710695114
DiMaio F, Leaver-Fay A, Bradley P, Baker D, André I. 2011. Modeling Symmetric Macromolecular Structures in Rosetta3. PLoS ONE 6:e20450. doi:10.1371/journal.pone.0020450
Férey G, Mellot-Draznieks C, Serre C, Millange F, Dutour J, Surblé S, Margiolaki I. 2005. A Chromium Teraphthalate-Based Solid with Unusually Large Pore Volumes and Surface Area. Science 309:2040-2042. doi:10.1126/science. 116275
Fleishman S J, Leaver-Fay A, Coin J E, Strauch E-M, Khare S D, Koga N, Ashworth J, Murphy P, Richter F, Lemmon G, Meiler J, Baker D. 2011. RosettaScripts: a scripting language interface to the Rosetta macromolecular modeling suite. PLoS ONE 6:e20161. doi:10.1371/journal.pone.0020161
Frederickson C J, Giblin L J, Krȩżel A, McAdoo D J, Muelle R N, Zeng Y, Balaji R V, Masalha R, Thompson R B, Fierke C A, Sarvey J M, de Valdenebro M, Prough D S, Zomow M H. 2006. Concentrations of extracellular free zinc (pZn)e in the central nervous system during simple anesthetization, ischemia and reperfusion. Experimental Neurology 198:285-293. doi:10.1016/j.expneurol.2005.08.030
Furukawa H, Ko N, Go Y B, Aratani N, Choi S B, Choi E, Yazaydin A Ö, Snurr R Q, O'Keeffe M, Kim J, Yaghi O M. 2010. Ultrahigh Porosity in Metal-Organic Frameworks. Science 329:424-428. doi:10.1126/science.1192160
Ghadiri M R, Granja J R, Milligan R A, McRee D E, Khazanovich N. 1993. Self-assembling organic nanotubes based on a cyclic peptide architecture. Nature 366:324-327. doi:10.1038/366324a0
Giuliano M W, Home W S, Gellman S H. 2009. An alpha/beta-peptide helix bundle with a pure beta3-amino acid core and a distinctive quaternary structure. J Am Chem Soc 131:9860-9861. doi:10.1021/ja8099294
Gonen S, DiMaio F, Gonen T, Baker D. 2015. Design of ordered two-dimensional arrays mediated by noncovalent protein-protein interfaces. Science 348:1365-1368. doi:10.1126/science.aaa9897
Hodgkin D C, Oughton B M. 1957. Possible molecular models for gramicidin S and their relationship to present ideas of protein structure. Biochem J 65:752-756. doi:10.1042/bj0650752
Home W S, Gellman S H. 2008. Foldamers with Heterogeneous Backbones. Acc Chem Res 41:1399-1408. doi:10.1021/ar800009n
Hosseinzadeh P, Bhardwaj G, Mulligan V K, Shortridge M D, Craven T W, Pardo-Avila F, Rettie S A, Kim D E, Silva D-A, Ibrahim Y M, Webb I K, Cort J R, Adkins J N, Varani G, Baker D. 2017. Comprehensive computational design of ordered peptide macrocycles. Science 358:1461-1466. doi:10.1126/science.aap7577
Hsia Y, Bale J B, Gonen S, Shi D, Sheffler W, Fong K K, Nattermann U, Xu C, Huang P-S, Ravichandran R, Yi S, Davis T N, Gonen T, King N P, Baker D. 2016. Design of a hyperstable 60-subunit protein icosahedron. Nature 535:136-139. doi:10.1038/nature18010
Hunt J B, Neece S H, Ginsburg A. 1985. The use of 4-(2-pyridylazo)resorcinol in studies of zinc release from Escherichia coli aspartate transcarbamoylase. Analytical Biochemistry 146:150-157. doi:10.1016/0003-2697(85)90409-9
King N P, Bale J B, Sheffler W, McNamara D E, Gonen S, Gonen T, Yeates T O, Baker D. 2014. Accurate design of co-assembling multi-component protein nanomaterials. Nature 510:103-108. doi:10.1038/nature13404
King N P, Sheffler W, Sawaya M R, Vollmar B S, Sumida J P, André I, Gonen T, Yeates T O, Baker D. 2012. Computational design of self-assembling protein nanomaterials with atomic level accuracy. Science 336:1171-1174. doi:10.1126/science.1219364
Leaver-Fay A, Tyka M, Lewis S M, Lange O F, Thompson J, Jacak R, Kaufman K W, Renfrew P D, Smith C A, Sheffler W, Davis I W, Cooper S, Treuille A, Mandell D J, Richter F, Ban Y-EA, Fleishman S J, Com J E, Kim D E, Lyskov S, Berrondo M, Mentzer S, Popović Z. Havranek J J, Karanicolas J, Das R, Meiler J, Kortemme T, Gray J J, Kuhlman B, Baker D, Bradley P. 2011. Rosetta3 Methods in Enzymology. Elsevier. pp. 545-574. doi:10.1016/B978-0-12-381270-4.00019-6
Mandal D, Nasrolahi Shirazi A, Parang K. 2014. Self-Assembly of Peptides to Nanostructures. Organic & biomolecular chemistry 12. doi:10.1039/c4ob00447g
Mandell D J, Coutsias E A, Kortemme T. 2009. Sub-angstrom accuracy in protein loop reconstruction by robotics-inspired conformational sampling. Nat Methods 6:551-552. doi:10.1038/nmeth0809-551
Maret W. 2017. Zinc in Cellular Regulation: The Nature and Significance of “Zinc Signals.” IJMS 18:2285. doi:10.3390/ijms18112285
Mulligan V K. 2020. The emerging role of computational design in peptide macrocycle drug discovery. Expert Opinion on Drug Discovery 15:833-852. doi:10.1080/17460441.2020.1751117
Mulligan V K, Kerman A, Ho S, Chakrabartty A. 2008. Denaturational stress induces formation of zinc-deficient monomers of Cu,Zn superoxide dismutase: implications for pathogenesis in amyotrophic lateral sclerosis. J Mol Biol 383:424-436. doi:10.1016/j.jmb.2008.08.024
Pintér Á, Haberhauer G. 2009. Synthesis of chiral threefold and sixfold functionalized macrocyclic imidazole peptides. Tetrahedron 65:2217-2225. doi:10.1016/j.tet.2009.01.047
Ranganathan D, Lakshmi C, Haridas V, Gopikumar M. 2000. Designer cyclopeptides for self-assembled tubular structures. Pure and applied chemistry 72:365-372.
Seo J S, Whang D, Lee H, Jun S I, Oh J, Jeon Y J, Kim K. 2000. A homochiral metal-organic porous material for enantioselective separation and catalysis. Nature 404:982-986. doi:10.1038/35010088
Slough D P, McHugh S M, Cummings A E, Dai P, Pentelute B L, Kritzer J A, Lin Y-S. 2018. Designing Well-Structured Cyclic Pentapeptides Based on Sequence-Structure Relationships. J Phys Chem B 122:3908-3919. doi:10.1021/acs.jpcb.8b01747
Strauch E-M, Bernard S M, La D, Bohn A J, Lee P S, Anderson C E, Nieusma T, Holstein C A, Garcia N K, Hooper K A, Ravichandran R, Nelson J W, Sheffler W, Bloom J D. Lee K K, Ward A B, Yager P, Fuller D H, Wilson I A, Baker D. 2017. Computational design of trimeric influenza-neutralizing proteins targeting the hemagglutinin receptor binding site. Nat Biotechnol 35:667-671. doi:10.1038/nbt.3907
van Maarseveen J H, Home W S, Ghadiri M R. 2005. Efficient Route to C2 Symmetric Heterocyclic Backbone Modified Cyclic Peptides. Org Lett 7:4503-4506. doi:10.1021/ol0518028
Witek J, Keller B G, Blatter M, Meissner A, Wagner T, Riniker S. 2016. Kinetic Models of Cyclosporin A in Polar and Apolar Environments Reveal Multiple Congruent Conformational States. J Chem Inf Model 56:1547-1562. doi:10.1021/acs.jcim.6b00251
Yeates T O. 2017. Geometric Principles for Designing Highly Symmetric Self-Assembling Protein Nanomaterials. Annu Rev Biophys 46:23-42. doi:10.1146/annurev-biophys-070816-033928
Yeates T O, Kent S B H. 2012. Racemic protein crystallography. Annu Rev Biophys 41:41-61. doi:10.1146/annurev-biophys-050511-102333

### Supplemental Materials and Methods

### Solid-Phase Peptide Synthesis and Purification

### Synthesis and On-Resin Cyclization of Peptides of Twelve Residues or Fewer

Those cyclic peptides that contained an acidic L-amino acid residue, and which had twelve amino acid residues or fewer, were synthesized using standard Fmoc solid-phase peptide synthesis (SPPS) on preloaded and sidechain-linked Fmoc-L-Asp(Wang resin LL)-ODmab or Fmoc-L-Glu(Wang resin LL)-ODmab resin. Linear, protected peptides were built on a CEM Liberty Blue™ Peptide Synthesizer with microwave heating at coupling and deprotection steps. After the final Fmoc deprotection, the resin was treated with 2% (v/v) hydrazine monohydrate in dimethylformamide (DMF) to remove the C-terminal Dmab protecting group; the N- and C-termini were then joined on-resin by a coupling reaction. A cleavage cocktail of TFA:Water:TIPS:DODT (92.5:2.5:2.5:2.5) was used for global deprotection of side-chains and to cleave the peptide from the resin. After the removal of residual TFA by evaporation, peptides were ether precipitated and further purified using RP-HPLC.

### Solution-Phase Cyclization of Larger Polypeptides

We found that cyclizing peptides longer than twelve residues on resin was challenging. In these cases the linear sequences were synthesized on the Liberty Blue N Peptide Synthesizer (with the C-terminus coupled to the resin) and then cyclized in-solution. Cl-TCP(Cl) resin from CEM was used as solid support, and the linear peptide cleaved from this resin without side-chain deprotection by treatment with 1% (v/v) TFA in dichloromethane (DCM). The protected peptide in DCM was drained into an equal volume of 50:50 acetonitrile and water. We evaporated the DCM using a rotovap apparatus, and the peptide in water and acetonitrile was then lyophilized. The resulting powder was redissolved in DCM to 1 mM based on synthesis scale assuming perfect efficiencies and 2 eq. of (7-Azabenzotriazol-1-yloxy)tripyrrolidinophosphonium hexafluorophosphate (PyAOP) was added directly to the solution. The solution was left on a magnetic stirrer for 30 minutes, then 10 eq. of N,N-diisopropylethylamine (DIEA) was added drop-wise and the cyclization reaction was left stirring overnight. As much of the solution as possible was evaporated, again using the rotovap apparatus, and the cyclic peptide was then deprotected, precipitated and purified as described herein.

### Peptide Purification

Crude peptides were purified using an Agilent Infinity™ Preparative high-pressure liquid chromatography (HPLC) system with an Agilent Zorbax™ SB-C18 column (9.4 mm×250 mm). We used a linear gradient of 1%/min for Solvent B (ACN with 0.1% TFA), and a flow rate of 5 mL/min, to purify peptides, with elution peaks detected using 214 nm absorbance. We confirmed mass and purity of peptides by electrospray ionization mass spectrometry (ESI-MS) on a Thermo Scientific TSQ Quantum Access™ mass spectrometer.

### X-Ray Crystallography

### Crystallization Conditions

All crystals were grown by hanging drop vapor diffusion. Equal volumes of peptide and reservoir solution (100 nL each) were combined using a robot and suspended over 100 μL of reservoir using robotics. Individual crystallization conditions for each peptide are as follows:

C2-1 and its mirror form were lyophilized in a 1:1 molar ratio and dissolved at a total peptide concentration of 25 mg/mL. The peptide crystallized in two different conditions, producing different structures. Although the peptides were grown from a racemic mixture, the first crystal form contained one hand only. This crystal form grew in space group P2₁2₁2₁ from a reservoir containing 0.1 M Citric acid pH 5.0 and 2.4 M ammonium sulfate. The crystal had a needle-like morphology with dimensions of 175×5×5 microns. The second crystal form grew in space group P1 from a reservoir containing 0.2M calcium chloride, 0.1 M HEPES pH 7.5, and 28% (w/v) PEG 400. Electron density shows evidence of epimerization (˜50%) at the DSER (residue 1) position. The crystal morphology was needle shaped, 200 microns long and less than 5 microns thick. Diffraction data from both crystal forms were collected at the Argonne National Laboratory Advanced Photon Source (APS), beamline 24-ID-E.

C3-1 and its mirror form were combined in a 1:1 molar ratio and dissolved in water for a total peptide concentration of 20 mg/mL. The peptide crystallized in two different conditions, yielding two crystal forms. The first crystal form grew in space group C2/c, from a reservoir composed of JCSG Core II A8 (0.1 M Tris pH 8.5, 5% (w/v) PEG 8000, 20% (w/v) PEG 300, 10% (w/v) Glycerol). The second crystal form grew in space group P1 from Morpheus screen condition C8, consisting of 0.09 M sodium nitrate, 0.09 M sodium phosphate dibasic, 0.09 M ammonium sulfate, 0.1 M HEPES, 0.1 M MOPS pH 7.5, 12.5% v/v 2-methyl-2,4-pentanediol, 12.5% PEG 1000, and 12.5% w/v PEG 3350. Diffraction data from both crystal forms were collected at the APS, beamline 24-ID-E.

C3-2 and its mirror form were combined in a 1:1 molar ratio and dissolved in water for a total peptide concentration of 22 mg/mL. The peptide crystallized in space group P3, from a reservoir composed of JCSG Core IV C5 (0.2 M Zinc acetate, 0.1 M imidazole pH 8.0, 2.5M Sodium chloride). Diffraction data were collected at the APS beamline 24-ID-C.

C3-3 and its mirror form were concentrated to 14.4 mg/mL each, for a total peptide concentration of 28.8 mg/mL. The peptide crystallized in the space group P31c, from a reservoir composed of JCSG Core III G10 (0.1 M Cadmium chloride, 0.1 M Sodium acetate pH 4.6, 30% (w/v) PEG 400). Diffraction data were collected at the APS on beamline APS 24-ID-C.

S2-1 was concentrated to 18.6 mg/mL. The reservoir contained 3.2 M ammonium sulfate and 0.1 M citrate, pH 5.0. The crystal morphology was rod shaped with dimensions 100×20×5 μm. Diffraction data were collected at the APS, beamline 24-ID-C using a wavelength of 0.8856 Å.

S2-2 was concentrated to 20.8 mg/mL. The reservoir contained 0.1 M potassium thiocyanate and 30% (w/v) polyethylene glycol (PEG) 2000 monomethylether (MME). For cryo-protection, the crystal was briefly immersed in a mixture of 65% reservoir and 35% ethylene glycol. Diffraction data were collected at the APS, beamline 24-ID-E using a wavelength of 0.9792 Å.

S2-3 was concentrated to 23.4 mg/mL. The peptide crystallized under two different conditions, producing different packings of space group P1. The first crystal form grew from a reservoir composed of 1.6 M tri-sodium citrate pH 6.5. The morphology was a trapezoidal plate (isosceles) with edges of 50×20×5 μm. The second crystal form grew from a reservoir composed of 0.2 M lithium sulfate, 0.1 M sodium acetate, and 50% (w/v) PEG 400. The morphology was pyramidal shaped with edges of approximately 30 μm. Diffraction data from both crystal forms were collected at the APS, beamline 24-ID-E using a wavelength of 0.9792 Å.

S2-4 was concentrated to 29.8 mg/mL. The peptide crystallized under two different conditions, producing different packings of space group P1. The first crystal form grew from a reservoir composed of 3.15 M ammonium sulfate and 0.1 M citric acid, pH 5.0. The morphology was a diamond shaped plate with edges of 100×100×20 sm. The second crystal form grew from a reservoir composed of 2.4 M sodium malonate, pH 7.0. The morphology was rod-like with edges of 400×50×50 μm. Diffraction data from both crystal forms were collected at the APS, beamline 24-ID-E.

S2-5 was concentrated to 27.4 mg/mL. The reservoir contained 0.17 M (NH₄)₂SO₄, 25.5% (w/v) PEG 4000, and 15% (v/v) glycerol. The morphology was rod-like with edges of 250×40×30 μm. Diffraction data were collected at the APS, beamline 24-ID-C

S2-6 was concentrated to 33.9 mg/mL. The reservoir contained 3.15 M ammonium sulfate, and 0.1 M citric acid, pH 5.0. The morphology was prismatic with edges of 100×80×60 μm. Diffraction data were collected at the APS, beamline 24-ID-C.

S4-1 was concentrated to 15 mg/mL in the presence of 100 mM zinc acetate. The peptide crystallized in holo and apo forms. The holo crystal form grew from a reservoir composed of 1.1 M sodium malonate dibasic monohydrate, 0.1 M HEPES pH 7.0, and 0.5% (v/v) Jeffamine-% ED-2003. The morphology was a square plate with edges of 100×100×40 μm. Diffraction data were collected at the APS, beamline 24-ID-C. The second crystal form grew from a reservoir composed of 3.15 M ammonium sulfate and 0.1 M citric acid, pH 5.0. The morphology was needle-like with edges of 150×5×5 μm. Diffraction data were collected at the APS, beamline 24-ID-E.

### X-Ray Diffraction and Data Analysis Protocol

X-ray diffraction data were collected at beamlines 24-ID-C and 24-ID-E at the Advanced Photon Source at Argonne National Laboratories as noted above for each crystal. Crystals were cooled to a temperature of 100 K. Diffraction data were indexed, integrated, scaled, and merged using the programs XDs/XSCALE or Denzo/scalepack (Kabsch, 2010; Otwinowski and Minor, 1997). Initial phases for all crystal structures were obtained by direct methods using the programs ShelxD or ShelxT (Sheldrick, 2015, 2008). Refinement was performed using the programs Rfmac5 or ShelX (Murshudov et al., 2011; Sheldrick, 2008). Model building was performed using the graphics program coot (Emsley et al., 2010).

### Metal Content and Affinity Assays

### Confirming Metal Content of Holo- and Apo-Polypeptides

We used a variant on the 4-(2-pyridylazo)resorcinol (PAR) assay described previously (Crow et al., 1997; Hunt et al., 1985; Mulligan et al., 2008) to confirm the zinc content of preparations of holo-S4-1 and holo-S4-2 polypeptides (incubated with 1.25 equivalents of ZnCl₂) or apo-S4-1 and apo-S4-2 polypeptides (solubilized using metal-free buffer following lyophilization after purification in 1% trifluoroacetic acid expected to prevent metal binding). To prevent trace metal contamination, all glassware was triple-rinsed with distilled water (MilliporeSigma, Burlington, Mass.), and Chelex resin (Bio-Rad, Hercules Calif.) was added to all stock buffers. A stock of 7.1 M guanidinium hydrochloride (Sigma-Aldrich, St. Louis, Mo.) was prepared in 100 mM HEPES, pH 7.5, and exact concentration was measured by refractometry (Nozaki, 1972). In 96-well plates (200 μl per well), 10 μM polypeptide was denatured in 6 M guanidine in the presence of 200 μM PAR. The change in absorbance at 490 nm on addition of polypeptide was monitored over time on a SpectraMAX™ M5e plate reader (Molecular Devices, San Jose, Calif.), and the amplitude of the change measured. To control for unbound metal, measurements were also made with buffer substituted for guanidine. Standard curves were prepared using ZnCl₂ to convert absorbance changes into molar concentrations of zinc released.

### Measuring Metal Affinity of 54-1 and S4-2 Polypeptides

We measured the affinity of the S4-1 and S4-2 polypeptide for zinc by competition with PAR. PAR binds zinc as a PAR₂Zn complex, which exhibits considerably enhanced absorbance near 490 nm as compared to free PAR. This provides a convenient means of measuring complex formation between the designed peptide and zinc through the resulting decrease in absorption at 490 nm. The dissociation constants of the PAR₂Zn complex were previously reported to be 1.8×10⁻⁶ M and 2.5×10⁻⁷ M for the first and second dissociation events, respectively (Hunt et al., 1985), allowing an approximate second-order dissociation constant of 4.5×10⁻¹³ M² to be computed. This in turn allows the dissociation constant for the polypeptide-Zn complex to be determined by competition. First, we write the expressions for the various dissociation constants as functions of concentrations of species:

$${K}_{d , pep} = \frac{{{[ pep ]}_{free} [ Zn ]}_{free}}{[ pep Zn ]} \qquad ( S4 )$$

$${K}_{d , P A {R}_{2} Zn} = \frac{{{[ P A R ]}_{free}^{2} [ Zn ]}_{free}}{[ P A {R}_{2} Zn ]} \qquad ( S5 )$$

$$We can also \text{write:}$$

$${[ pep ]}_{total} = [ pep Zn ] + {[ pep ]}_{free} \qquad \text{(S6)}$$

$${[ P A R ]}_{total} = 2 [ P A {R}_{2} Zn ] + {[ P A R ]}_{free} \qquad \text{(S7)}$$

The above four equations can be combined to yield:

$$\frac{{K}_{d , pep} [ pep Zn ]}{{[ pep ]}_{total} - [ pep Zn ]} = \frac{{K}_{d , P A {R}_{2} Zn} [ P A {R}_{2} Zn ]}{{( {[ P A R ]}_{total} - 2 [ P A {R}_{2} Zn ] )}^{2}} \qquad ( S8 )$$

Since we are working at concentrations well above the K_{d} of the PAR₂Zn complex, we can make the approximation that all zinc is bound:

[Zn]ₜₒₜₐₗ=[Zn]_{free}+[pepZn]+[PAR₂Zn]≈[pepZn]+[PAR₂Zn] (S9)

Rearranging for [pepZn] and substituting into Eq. S8, we obtain an expression in which all other values are either known, set by the experimenter, or measured from the absorbance, and the sole unknown value is K_{d,pep}:

$$\frac{{K}_{d , pep} ( {[ Zn ]}_{total} - [ P A {R}_{2} Zn ] )}{{[ pep ]}_{total} - {[ Zn ]}_{total} + [ P A {R}_{2} Zn ]} = \frac{{K}_{d , P A {R}_{2} Zn} [ P A {R}_{2} Zn ]}{{( {[ P A R ]}_{total} - 2 [ P A {R}_{2} Zn ] )}^{2}} \qquad ( S10 )$$

Binding data can be fitted to the expression above numerically. Alternatively, analytic solvers can be used to produce an unwieldy but exact expression for [PAR₂Zn] as a function of polypeptide concentration (which is what we did, using Maple™ software [Maplesoft, Waterloo, ON, Canada]), to which titration data may be fitted to obtain K_{d,pep}. Our fits were performed with Origin software (OriginLab, Northampton, Mass., USA).

Experimentally, 0 to 50 μM apo-S4-1 or apo-S4-2 polypeptide was incubated with 10 μM ZnCl₂ and 500 μM PAR in 100 mM HEPES buffer, pH 7.5, in a 96-well plate. Total solution volume was 100 μl. Again, glassware was triple-rinsed with distilled water, and Chelex resin was added to all stock solutions. Absorbance was monitored at 490 nm using a SpectraMAX M5e plate reader, and signals were averaged for several minutes after reaching plateaux. Standard curves using ZnCl₂ in PAR were used to convert absorbance readings to [PAR₂Zn].

### SUPPLEMENTARY REFERENCES

Alford R F, Leaver-Fay A, Jeliazkov J R, O'Meara M J, DiMaio F P, Park H, Shapovalov M V, Renfrew P D, Mulligan V K, Kappel K, Labonte J W, Pacella M S, Bonneau R, Bradley P, Dunbrack R L, Das R, Baker D, Kuhlman B, Kortemme T, Gray J J. 2017. The Rosetta All-Atom Energy Function for Macromolecular Modeling and Design. Journal of Chemical Theory and Computation 13:3031-3048. doi:10.1021/acs.jctc.7b00125
André I, Bradley P, Wang C, Baker D. 2007. Prediction of the structure of symmetrical protein assemblies. Proc Natl Acad Sci USA 104:17656-17661. doi:10.1073/pnas.0702626104
Bhardwaj G, Mulligan V K, Bahl C D, Gilmore J M, Harvey P J, Cheneval O, Buchko G W, Pulavarti SVSRK, Kaas Q, Eletsky A, Huang P-S, Johnsen W A, Greisen P J, Rocklin G J, Song Y, Linsky T W, Watkins A, Rettie S A, Xu X, Carter L P, Bonneau R, Olson J M, Coutsias E, Correnti C E, Szyperski T, Craik D J, Baker D. 2016. Accurate de novo design of hyperstable constrained peptides. Nature 538:329-335.
Burnside W. 1900. On Some Properties of Groups of Odd Order. Proceedings of the London Mathematical Society, 33:162-184.
Coutsias E A, Seok C, Jacobson M P, Dill K A. 2004. A kinematic view of loop closure. J Comput Chem 25:510-528. doi:10.1002/jcc.10416
Crow J P, Sampson J B, Zhuang Y. Thompson J A, Beckman J S. 1997. Decreased zinc affinity of amyotrophic lateral sclerosis-associated superoxide dismutase mutants leads to enhanced catalysis of tyrosine nitration by peroxynitrite. J Neurochem 69:1936-1944. doi:10.1046/j.1471-4159.1997.69051936.x
Dang B, Wu H, Mulligan V K, Mravic M, Wu Y, Lemmin T, Ford A, Silva D-A, Baker D, DeGrado W F. 2017. De novo design of covalently constrained mesosize protein scaffolds with unique tertiary structures. Proceedings of the National Academy of Sciences 114:10852-10857. doi:10.1073/pnas.1710695114
DiMaio F, Leaver-Fay A, Bradley P, Baker D, André I. 2011. Modeling Symmetric Macromolecular Structures in Rosetta3. PLoS ONE 6:e20450. doi:10.1371/journal.pone.0020450
Drew K, Renfrew P D, Craven T W, Butterfoss G L, Chou F-C, Lyskov S, Bullock B N, Watkins A, Labonte J W, Pacella M, Kilambi K P, Leaver-Fay A, Kuhlman B, Gray J J, Bradley P, Kirshenbaum K, Arora P S, Das R, Bonneau R. 2013. Adding Diverse Noncanonical Backbones to Rosetta: Enabling Peptidomimetic Design. PLoS ONE 8:e67051. doi:10.1371/journal.pone.0067051
Emsley P, Lohkamp B, Scott W G, Cowtan K. 2010. Features and development of Coot. Acta Crystallogr D Biol Crystallogr 66:486-501. doi:10.1107/S1907444910007493
Fleishman S J, Leaver-Fay A, Corn J E, Strauch E-M, Khare S D, Koga N, Ashworth J, Murphy P, Richter F, Lemmon G, Meiler J, Baker D. 2011. RosettaScripts: a scripting language interface to the Rosetta macromolecular modeling suite. PLoS ONE 6:e20161. doi:10.1371/journal.pone.0020161
Hosseinzadeh P, Bhardwaj G, Mulligan V K, Shortridge M D, Craven T W, Pardo-Avila F, Rettie S A, Kim D E, Silva D-A, Ibrahim Y M, Webb I K, Cort J R, Adkins J N, Varani G, Baker D. 2017. Comprehensive computational design of ordered peptide macrocycles. Science 358:1461-1466. doi:10.1126/science.aap7577
Hunt J B, Neece S H, Ginsburg A. 1985. The use of 4-(2-pyridylazo)resorcinol in studies of zinc release from Escherichia coli aspartate transcarbamoylase. Analytical Biochemistry 146:150-157. doi:10.1016/0003-2697(85)90409-9
Kabsch W. 2010. XDS. Acta Crystallogr D Biol Crystallogr 66:125-132. doi:10.1107/S0907444909047337
Leaver-Fay A, Tyka M, Lewis S M, Lange O F, Thompson J, Jacak R, Kaufman K W, Renfrew P D, Smith C A, Sheffler W, Davis I W, Cooper S, Treuille A, Mandell D J, Richter F, Ban Y-EA, Fleishman S J, Com J E, Kim D E, Lyskov S, Berrondo M, Mentzer S, Popović Z, Havranek J, Karanicolas J, Das R, Meiler J, Kortemme T, Gray J J, Kuhlman B, Baker D, Bradley P. 2011. Rosetta3 Methods in Enzymology. Elsevier. pp. 545-574. doi:10.1016/B978-0-12-381270-4.00019-6
Mandell D J, Coutsias E A, Kortemme T. 2009. Sub-angstrom accuracy in protein loop reconstruction by robotics-inspired conformational sampling. Nat Methods 6:551-552. doi:10.1038/nmeth0809-551
Mulligan V K, Kerman A, Ho S, Chakrabartty A. 2008. Denaturational stress induces formation of zinc-deficient monomers of Cu,Zn superoxide dismutase: implications for pathogenesis in amyotrophic lateral sclerosis. J Mol Biol 383:424-436. doi:10.1016/j.jmb.2008.08.024
Murshudov G N, Skubák P, Lebedev A A, Pannu N S, Steiner R A, Nicholls R A, Winn M D, Long F, Vagin A A. 2011. REFMAC 5 for the refinement of macromolecular crystal structures. Acta Crystallogr D Biol Crystallogr 67:355-367. doi:10.1107/S0907444911001314
Nozaki Y. 1972. The preparation of guanidine hydrochloride Methods in Enzymology, Enzyme Structure, Part C. Academic Press. pp. 43-50. doi:10.1016/S0076-6879(72)26005-0
Otwinowski Z, Minor W. 1997. Processing of X-ray diffraction data collected in oscillation mode Methods in Enzymology, Macromolecular Crystallography Part A. Academic Press. pp. 307-326. doi:10.1016/S0076-6879(97)76066-X
Renfrew P D, Choi E J, Bonneau R, Kuhlman B. 2012. Incorporation of Noncanonical Amino Acids into Rosetta and Use in Computational Protein-Peptide Interface Design. PLoS ONE 7:e32637. doi:10.1371/journal.pone.0032637
Sheldrick G M. 2015. SHELXT—integrated space-group and crystal-structure determination. Acta Crystallogr A Found Adv 71:3-8. doi:10.1107/S2053273314026370
Sheldrick G M. 2008. A short history of SHELX. Acta Crystallogr, A, Found Crystallogr 64:112-122. doi:10.1107/S0108767307043930
Tamames B, Sousa S F, Tamames J, Fernandes P A, Ramos M J. 2007. Analysis of zinc-ligand bond lengths in metalloproteins: Trends and patterns. Proteins: Structure, Function, and Bioinformatics 69:466-475. doi:10.1002/prot.21536
Tange O. 2018. GNU Parallel 2018. Ole Tange. doi:10.5281/zenodo.1146014
Tange O. 2011. GNU Parallel: The Command-Line Power Tool. The USENIX Magazine 42-47.

1. A polypeptide comprising or consisting of an amino acid sequence at least 66% identical to the amino acid sequence selected from the group consisting of SEQ ID NO: 1-91 as shown in Table 1, wherein:
(a) amino acid residues in upper case are L amino acids, and residues in lower case are D amino acids;
(b) X is 2-aminoisobutyric acid (AIB);
(c) no amino acid changes at proline or AIB residues in the reference peptide are permitted; and
(d) any amino acid changes must maintain chirality relative to the reference peptide.

2. The polypeptide of claim 1, wherein no proline or AIB residues may be added by amino acid change relative to the reference polypeptide.

3. The polypeptide of claim 1, wherein the polypeptide has C2 symmetry, and comprises an amino acid sequence at least 66% identical to the amino acid sequence selected from the group consisting of SEQ ID NO:1-8.

4. The polypeptide of claim 1, wherein the polypeptide has S2 symmetry, and comprises an amino acid sequence at least 66% identical to the amino acid sequence selected from the group consisting of SEQ ID NO: 9-14.

5. The polypeptide of claim 1, wherein the polypeptide has C3 symmetry, and comprises an amino acid sequence at least 66% identical to the amino acid sequence selected from the group consisting of SEQ ID NO:15-72.

6. The polypeptide of claim 5, wherein when the first residue of the asymmetric unit is L-Proline and the third residue of the asymmetric unit is L-Aspartic acid, the 2nd residue can be any non-glycine, non-proline, non-AIB L-amino acid

7. The polypeptide of claim 1, wherein the polypeptide has C4 symmetry, and comprises an amino acid sequence at least 66% identical to the amino acid sequence selected from the group consisting of SEQ ID NO:73-76.

8. The polypeptide of claim 1, wherein the polypeptide has C5 symmetry, and comprises an amino acid sequence at least 66% identical to the amino acid sequence selected from the group consisting of SEQ ID NO:77-90.

9. The polypeptide of claim 1, wherein the polypeptide has S4 symmetry, and comprises an amino acid sequence at least 66% identical to the amino acid sequence of SEQ ID NO:91.

10. The polypeptide of claim 7, wherein the polypeptide is bound to a metal ion, including but not limited to Zn2+.

11. The polypeptide of claim 1, wherein the polypeptide is conjugated to one or more additional components.

12. The polypeptide of claim 11, where the one or more additional components are selected from the group consisting of detectable tags, small molecules, radioactive agents, antibodies, polyethylene glycol, therapeutic moieties, and diagnostic moieties.

13. A method for using the polypeptide of claim 1 any use described herein, such as assembling with metals to form super molecular crystals.

14. A method for designing mixed chirality peptide macrocycles with internal symmetry according to any embodiment or combination of embodiments described herein.

15. A nucleic acid encoding a polypeptide comprising or consisting of an amino acid sequence at least 66% identical to the amino acid sequence selected from the group consisting of SEQ ID NO: 15, 17, 29, 47, 49, 67, 69, 71, 77, and 81, wherein:
(a) amino acid residues in upper case are L amino acids;
(b) no amino acid changes at proline residues in the reference peptide are permitted; and
(d) any amino acid changes must maintain chirality relative to the reference peptide.

16. An expression vector comprising the nucleic acid of claim 15 operatively linked to a control sequence.

17. A host cell comprising the expression vector of claim 16.


SEQUENCE LISTING (91 sequences)

SEQ ID NO: 1 - protein, 10 aa, Artificial Sequence
  Synthetic
  MISC_FEATURE (1)..(2): D enantiomer
  MOD_RES (3)..(3): Aib
  MISC_FEATURE (6)..(7): D enantiomer
  MOD_RES (8)..(8): Aib
SEXSLSEXSL

SEQ ID NO: 2 - protein, 10 aa, Artificial Sequence
  Synthetic
  MOD_RES (3)..(3): Aib
  MISC_FEATURE (4)..(5): D enantiomer
  MOD_RES (8)..(8): Aib
  MISC_FEATURE (9)..(10): D enantiomer
SEXSLSEXSL

SEQ ID NO: 3 - protein, 10 aa, Artificial Sequence
  Synthetic
  MISC_FEATURE (1)..(2): D enantiomer
  MISC_FEATURE (6)..(7): D enantiomer
YPDQSYPDQS

SEQ ID NO: 4 - protein, 10 aa, Artificial Sequence
  Synthetic
  MISC_FEATURE (3)..(5): D enantiomer
  MISC_FEATURE (8)..(10): D enantiomer
YPDQSYPDQS

SEQ ID NO: 5 - protein, 10 aa, Artificial Sequence
  Synthetic
  MOD_RES (2)..(2): Aib
  MISC_FEATURE (5)..(5): D enantiomer
  MOD_RES (7)..(7): Aib
  MISC_FEATURE (10)..(10): D enantiomer
DXRLQDXRLQ

SEQ ID NO: 6 - protein, 10 aa, Artificial Sequence
  Synthetic
  MISC_FEATURE (1)..(1): D enantiomer
  MOD_RES (2)..(2): Aib
  MISC_FEATURE (3)..(4): D enantiomer
  MISC_FEATURE (6)..(6): D enantiomer
  MOD_RES (7)..(7): Aib
  MISC_FEATURE (8)..(9): D enantiomer
DXRLQDXRLQ

SEQ ID NO: 7 - protein, 12 aa, Artificial Sequence
  Synthetic
  MISC_FEATURE (3)..(5): D enantiomer
  MISC_FEATURE (9)..(11): D enantiomer
PTEEYQPTEEYQ

SEQ ID NO: 8 - protein, 12 aa, Artificial Sequence
  Synthetic
  MISC_FEATURE (1)..(2): D enantiomer
  MISC_FEATURE (6)..(8): D enantiomer
PTEEYQPTEEYQ

SEQ ID NO: 9 - protein, 8 aa, Artificial Sequence
  Synthetic
  MISC_FEATURE (2)..(2): D enantiomer
  MISC_FEATURE (4)..(5): D enantiomer
  MISC_FEATURE (7)..(7): D enantiomer
PEVKPEVK

SEQ ID NO: 10 - protein, 8 aa, Artificial Sequence
  Synthetic
  MISC_FEATURE (2)..(2): D enantiomer
  MISC_FEATURE (4)..(5): D enantiomer
  MISC_FEATURE (7)..(7): D enantiomer
PKVEPKVE

SEQ ID NO: 11 - protein, 10 aa, Artificial Sequence
  Synthetic
  MISC_FEATURE (1)..(1): D enantiomer
  MISC_FEATURE (7)..(10): D enantiomer
QTRPDQTRPD

SEQ ID NO: 12 - protein, 10 aa, Artificial Sequence
  Synthetic
  MISC_FEATURE (2)..(3): D enantiomer
  MISC_FEATURE (5)..(6): D enantiomer
EPPKVEPPKV

SEQ ID NO: 13 - protein, 10 aa, Artificial Sequence
  Synthetic
  MISC_FEATURE (1)..(1): D enantiomer
  MISC_FEATURE (4)..(4): D enantiomer
  MISC_FEATURE (7)..(8): D enantiomer
  MISC_FEATURE (10)..(10): D enantiomer
PSEIRPSEIR

SEQ ID NO: 14 - protein, 12 aa, Artificial Sequence
  Synthetic
  MISC_FEATURE (1)..(1): D enantiomer
  MISC_FEATURE (3)..(4): D enantiomer
  MISC_FEATURE (6)..(6): D enantiomer
  MISC_FEATURE (8)..(8): D enantiomer
  MISC_FEATURE (11)..(11): D enantiomer
ANKHPEANKHPE

SEQ ID NO: 15 - protein, 9 aa, Artificial Sequence
  Synthetic
PRDPRDPRD

SEQ ID NO: 16 - protein, 9 aa, Artificial Sequence
  Synthetic
  MISC_FEATURE (1)..(9): D enantiomer
PRDPRDPRD

SEQ ID NO: 17 - protein, 9 aa, Artificial Sequence
  Synthetic
PDDPDDPDD

SEQ ID NO: 18 - protein, 9 aa, Artificial Sequence
  Synthetic
  MISC_FEATURE (1)..(9): D enantiomer
PDDPDDPDD

SEQ ID NO: 19 - protein, 12 aa, Artificial Sequence
  Synthetic
  MISC_FEATURE (3)..(3): D enantiomer
  MISC_FEATURE (7)..(7): D enantiomer
  MISC_FEATURE (11)..(11): D enantiomer
PRVDPRVDPRVD

SEQ ID NO: 20 - protein, 12 aa, Artificial Sequence
  Synthetic
  MISC_FEATURE (1)..(2): D enantiomer
  MISC_FEATURE (4)..(6): D enantiomer
  MISC_FEATURE (8)..(10): D enantiomer
  MISC_FEATURE (12)..(12): D enantiomer
PRVDPRVDPRVD

SEQ ID NO: 21 - protein, 9 aa, Artificial Sequence
  Synthetic
  MISC_FEATURE (1)..(2): D enantiomer
  MISC_FEATURE (4)..(5): D enantiomer
  MISC_FEATURE (7)..(8): D enantiomer
DLKDLKDLK

SEQ ID NO: 22 - protein, 9 aa, Artificial Sequence
  Synthetic
  MISC_FEATURE (3)..(3): D enantiomer
  MISC_FEATURE (6)..(6): D enantiomer
  MISC_FEATURE (9)..(9): D enantiomer
DLKDLKDLK

SEQ ID NO: 23 - protein, 12 aa, Artificial Sequence
  Synthetic
  MISC_FEATURE (2)..(2): D enantiomer
  MISC_FEATURE (4)..(4): D enantiomer
  MISC_FEATURE (6)..(6): D enantiomer
  MISC_FEATURE (8)..(8): D enantiomer
  MISC_FEATURE (10)..(10): D enantiomer
  MISC_FEATURE (12)..(12): D enantiomer
DPYKDPYKDPYK

SEQ ID NO: 24 - protein, 12 aa, Artificial Sequence
  Synthetic
  MISC_FEATURE (1)..(1): D enantiomer
  MISC_FEATURE (3)..(3): D enantiomer
  MISC_FEATURE (5)..(5): D enantiomer
  MISC_FEATURE (7)..(7): D enantiomer
  MISC_FEATURE (9)..(9): D enantiomer
  MISC_FEATURE (11)..(11): D enantiomer
DPYKDPYKDPYK

SEQ ID NO: 25 - protein, 12 aa, Artificial Sequence
  Synthetic
  MISC_FEATURE (2)..(2): D enantiomer
  MISC_FEATURE (4)..(4): D enantiomer
  MISC_FEATURE (6)..(6): D enantiomer
  MISC_FEATURE (8)..(8): D enantiomer
  MISC_FEATURE (10)..(10): D enantiomer
  MISC_FEATURE (12)..(12): D enantiomer
DPKYDPKYDPKY

SEQ ID NO: 26 - protein, 12 aa, Artificial Sequence
  Synthetic
  MISC_FEATURE (1)..(1): D enantiomer
  MISC_FEATURE (3)..(3): D enantiomer
  MISC_FEATURE (5)..(5): D enantiomer
  MISC_FEATURE (7)..(7): D enantiomer
  MISC_FEATURE (9)..(9): D enantiomer
  MISC_FEATURE (11)..(11): D enantiomer
DPKYDPKYDPKY

SEQ ID NO: 27 - protein, 12 aa, Artificial Sequence
  Synthetic
  MISC_FEATURE (2)..(4): D enantiomer
  MISC_FEATURE (6)..(8): D enantiomer
  MISC_FEATURE (10)..(12): D enantiomer
DVPKDVPKDVPK

SEQ ID NO: 28 - protein, 12 aa, Artificial Sequence
  Synthetic
  MISC_FEATURE (1)..(1): D enantiomer
  MISC_FEATURE (5)..(5): D enantiomer
  MISC_FEATURE (9)..(9): D enantiomer
DVPKDVPKDVPK

SEQ ID NO: 29 - protein, 9 aa, Artificial Sequence
  Synthetic
EPKEPKEPK

SEQ ID NO: 30 - protein, 9 aa, Artificial Sequence
  Synthetic
  MISC_FEATURE (1)..(9): D enantiomer
EPKEPKEPK

SEQ ID NO: 31 - protein, 12 aa, Artificial Sequence
  Synthetic
  MISC_FEATURE (3)..(3): D enantiomer
  MISC_FEATURE (7)..(7): D enantiomer
  MISC_FEATURE (11)..(11): D enantiomer
DPRYDPRYDPRY

SEQ ID NO: 32 - protein, 12 aa, Artificial Sequence
  Synthetic
  MISC_FEATURE (1)..(2): D enantiomer
  MISC_FEATURE (4)..(6): D enantiomer
  MISC_FEATURE (8)..(10): D enantiomer
  MISC_FEATURE (12)..(12): D enantiomer
DPRYDPRYDPRY

SEQ ID NO: 33 - protein, 12 aa, Artificial Sequence
  Synthetic
  MISC_FEATURE (2)..(2): D enantiomer
  MISC_FEATURE (6)..(6): D enantiomer
  MISC_FEATURE (10)..(10): D enantiomer
EKAPEKAPEKAP

SEQ ID NO: 34 - protein, 12 aa, Artificial Sequence
  Synthetic
  MISC_FEATURE (1)..(1): D enantiomer
  MISC_FEATURE (3)..(5): D enantiomer
  MISC_FEATURE (7)..(9): D enantiomer
  MISC_FEATURE (11)..(12): D enantiomer
EKAPEKAPEKAP

SEQ ID NO: 35 - protein, 15 aa, Artificial Sequence
  Synthetic
  MISC_FEATURE (2)..(3): D enantiomer
  MISC_FEATURE (7)..(8): D enantiomer
  MISC_FEATURE (12)..(13): D enantiomer
KDPNLKDPNLKDPNL

SEQ ID NO: 36 - protein, 15 aa, Artificial Sequence
  Synthetic
  MISC_FEATURE (1)..(1): D enantiomer
  MISC_FEATURE (4)..(6): D enantiomer
  MISC_FEATURE (9)..(11): D enantiomer
  MISC_FEATURE (14)..(15): D enantiomer
KDPNLKDPNLKDPNL

SEQ ID NO: 37 - protein, 15 aa, Artificial Sequence
  Synthetic
  MISC_FEATURE (2)..(4): D enantiomer
  MISC_FEATURE (7)..(8): D enantiomer
  MISC_FEATURE (12)..(13): D enantiomer
AVDQKAVDQKAVDQK

SEQ ID NO: 38 - protein, 15 aa, Artificial Sequence
  Synthetic
  MISC_FEATURE (1)..(1): D enantiomer
  MISC_FEATURE (4)..(6): D enantiomer
  MISC_FEATURE (9)..(11): D enantiomer
  MISC_FEATURE (14)..(15): D enantiomer
AVDQKAVDQKAVDQK

SEQ ID NO: 39 - protein, 15 aa, Artificial Sequence
  Synthetic
  MISC_FEATURE (1)..(4): D enantiomer
  MISC_FEATURE (6)..(9): D enantiomer
  MISC_FEATURE (11)..(14): D enantiomer
DLQKPDLQKPDLQKP

SEQ ID NO: 40 - protein, 15 aa, Artificial Sequence
  Synthetic
  MISC_FEATURE (5)..(5): D enantiomer
  MISC_FEATURE (10)..(10): D enantiomer
  MISC_FEATURE (15)..(15): D enantiomer
DLQKPDLQKPDLQKP

SEQ ID NO: 41 - protein, 9 aa, Artificial Sequence
  Synthetic
  MISC_FEATURE (2)..(3): D enantiomer
  MISC_FEATURE (5)..(6): D enantiomer
  MISC_FEATURE (8)..(9): D enantiomer
DLKDLKDLK

SEQ ID NO: 42 - protein, 9 aa, Artificial Sequence
  Synthetic
  MISC_FEATURE (1)..(1): D enantiomer
  MISC_FEATURE (4)..(4): D enantiomer
  MISC_FEATURE (7)..(7): D enantiomer
DLKDLKDLK

SEQ ID NO: 43 - protein, 12 aa, Artificial Sequence
  Synthetic
  MISC_FEATURE (1)..(3): D enantiomer
  MISC_FEATURE (5)..(7): D enantiomer
  MISC_FEATURE (9)..(11): D enantiomer
PVRDPVRDPVRD

SEQ ID NO: 44 - protein, 12 aa, Artificial Sequence
  Synthetic
  MISC_FEATURE (4)..(4): D enantiomer
  MISC_FEATURE (8)..(8): D enantiomer
  MISC_FEATURE (12)..(12): D enantiomer
PVRDPVRDPVRD

SEQ ID NO: 45 - protein, 12 aa, Artificial Sequence
  Synthetic
  MISC_FEATURE (2)..(2): D enantiomer
  MISC_FEATURE (6)..(6): D enantiomer
  MISC_FEATURE (10)..(10): D enantiomer
REPIREPIREPI

SEQ ID NO: 46 - protein, 12 aa, Artificial Sequence
  Synthetic
  MISC_FEATURE (1)..(1): D enantiomer
  MISC_FEATURE (3)..(5): D enantiomer
  MISC_FEATURE (7)..(9): D enantiomer
  MISC_FEATURE (11)..(12): D enantiomer
REPIREPIREPI

SEQ ID NO: 47 - protein, 12 aa, Artificial Sequence
  Synthetic
KPVDKPVDKPVD

SEQ ID NO: 48 - protein, 12 aa, Artificial Sequence
  Synthetic
  MISC_FEATURE (1)..(12): D enantiomer
KPVDKPVDKPVD

SEQ ID NO: 49 - protein, 12 aa, Artificial Sequence
  Synthetic
DKTLDKTLDKTL

SEQ ID NO: 50 - protein, 12 aa, Artificial Sequence
  Synthetic
  MISC_FEATURE (1)..(12): D enantiomer
DKTLDKTLDKTL

SEQ ID NO: 51 - protein, 12 aa, Artificial Sequence
  Synthetic
  MISC_FEATURE (1)..(1): D enantiomer
  MISC_FEATURE (3)..(3): D enantiomer
  MISC_FEATURE (5)..(5): D enantiomer
  MISC_FEATURE (7)..(7): D enantiomer
  MISC_FEATURE (9)..(9): D enantiomer
  MISC_FEATURE (11)..(11): D enantiomer
DPYRDPYRDPYR

SEQ ID NO: 52 - protein, 12 aa, Artificial Sequence
  Synthetic
  MISC_FEATURE (2)..(2): D enantiomer
  MISC_FEATURE (4)..(4): D enantiomer
  MISC_FEATURE (6)..(6): D enantiomer
  MISC_FEATURE (8)..(8): D enantiomer
  MISC_FEATURE (10)..(10): D enantiomer
  MISC_FEATURE (12)..(12): D enantiomer
DPYRDPYRDPYR

SEQ ID NO: 53 - protein, 12 aa, Artificial Sequence
  Synthetic
  MISC_FEATURE (3)..(3): D enantiomer
  MISC_FEATURE (7)..(7): D enantiomer
  MISC_FEATURE (11)..(11): D enantiomer
DKVYDKVYDKVY

SEQ ID NO: 54 - protein, 12 aa, Artificial Sequence
  Synthetic
  MISC_FEATURE (1)..(2): D enantiomer
  MISC_FEATURE (4)..(6): D enantiomer
  MISC_FEATURE (8)..(10): D enantiomer
  MISC_FEATURE (12)..(12): D enantiomer
DKVYDKVYDKVY

SEQ ID NO: 55 - protein, 12 aa, Artificial Sequence
  Synthetic
  MISC_FEATURE (2)..(2): D enantiomer
  MISC_FEATURE (4)..(4): D enantiomer
  MISC_FEATURE (6)..(6): D enantiomer
  MISC_FEATURE (8)..(8): D enantiomer
  MISC_FEATURE (10)..(10): D enantiomer
  MISC_FEATURE (12)..(12): D enantiomer
DLRSDLRSDLRS

SEQ ID NO: 56 - protein, 12 aa, Artificial Sequence
  Synthetic
  MISC_FEATURE (1)..(1): D enantiomer
  MISC_FEATURE (3)..(3): D enantiomer
  MISC_FEATURE (5)..(5): D enantiomer
  MISC_FEATURE (7)..(7): D enantiomer
  MISC_FEATURE (9)..(9): D enantiomer
  MISC_FEATURE (11)..(11): D enantiomer
DLRSDLRSDLRS

SEQ ID NO: 57 - protein, 9 aa, Artificial Sequence
  Synthetic
  MISC_FEATURE (2)..(2): D enantiomer
  MISC_FEATURE (5)..(5): D enantiomer
  MISC_FEATURE (8)..(8): D enantiomer
PERPERPER

SEQ ID NO: 58 - protein, 9 aa, Artificial Sequence
  Synthetic
  MISC_FEATURE (1)..(1): D enantiomer
  MISC_FEATURE (3)..(4): D enantiomer
  MISC_FEATURE (6)..(7): D enantiomer
  MISC_FEATURE (9)..(9): D enantiomer
PERPERPER

SEQ ID NO: 59 - protein, 15 aa, Artificial Sequence
  Synthetic
  MISC_FEATURE (3)..(3): D enantiomer
  MISC_FEATURE (5)..(5): D enantiomer
  MISC_FEATURE (8)..(8): D enantiomer
  MISC_FEATURE (10)..(10): D enantiomer
  MISC_FEATURE (13)..(13): D enantiomer
  MISC_FEATURE (15)..(15): D enantiomer
PQKDVPQKDVPQKDV

SEQ ID NO: 60 - protein, 15 aa, Artificial Sequence
  Synthetic
  MISC_FEATURE (1)..(2): D enantiomer
  MISC_FEATURE (4)..(4): D enantiomer
  MISC_FEATURE (6)..(7): D enantiomer
  MISC_FEATURE (9)..(9): D enantiomer
  MISC_FEATURE (11)..(12): D enantiomer
  MISC_FEATURE (14)..(15): D enantiomer
PQKDVPQKDVPQKDV

SEQ ID NO: 61 - protein, 18 aa, Artificial Sequence
  Synthetic
  MISC_FEATURE (1)..(2): D enantiomer
  MISC_FEATURE (7)..(8): D enantiomer
  MISC_FEATURE (13)..(14): D enantiomer
LPEASKLPEASKLPEASK

SEQ ID NO: 62 - protein, 18 aa, Artificial Sequence
  Synthetic
  MISC_FEATURE (3)..(6): D enantiomer
  MISC_FEATURE (9)..(12): D enantiomer
  MISC_FEATURE (15)..(18): D enantiomer
LPEASKLPEASKLPEASK

SEQ ID NO: 63 - protein, 12 aa, Artificial Sequence
  Synthetic
  MISC_FEATURE (1)..(1): D enantiomer
  MISC_FEATURE (3)..(5): D enantiomer
  MISC_FEATURE (7)..(9): D enantiomer
  MISC_FEATURE (11)..(12): D enantiomer
IKEPIKEPIKEP

SEQ ID NO: 64 - protein, 12 aa, Artificial Sequence
  Synthetic
  MISC_FEATURE (2)..(2): D enantiomer
  MISC_FEATURE (6)..(6): D enantiomer
  MISC_FEATURE (10)..(10): D enantiomer
IKEPIKEPIKEP

SEQ ID NO: 65 - protein, 15 aa, Artificial Sequence
  Synthetic
  MISC_FEATURE (5)..(5): D enantiomer
  MISC_FEATURE (10)..(10): D enantiomer
  MISC_FEATURE (15)..(15): D enantiomer
PTKDVPTKDVPTKDV

SEQ ID NO: 66 - protein, 15 aa, Artificial Sequence
  Synthetic
  MISC_FEATURE (1)..(4): D enantiomer
  MISC_FEATURE (6)..(9): D enantiomer
  MISC_FEATURE (11)..(14): D enantiomer
PTKDVPTKDVPTKDV

SEQ ID NO: 67 - protein, 9 aa, Artificial Sequence
  Synthetic
PEIPEIPEI

SEQ ID NO: 68 - protein, 9 aa, Artificial Sequence
  Synthetic
  MISC_FEATURE (1)..(9): D enantiomer
PEIPEIPEI

SEQ ID NO: 69 - protein, 9 aa, Artificial Sequence
  Synthetic
PHIPHIPHI

SEQ ID NO: 70 - protein, 9 aa, Artificial Sequence
  Synthetic
  MISC_FEATURE (1)..(9): D enantiomer
PHIPHIPHI

SEQ ID NO: 71 - protein, 9 aa, Artificial Sequence
  Synthetic
PNDPNDPND

SEQ ID NO: 72 - protein, 9 aa, Artificial Sequence
  Synthetic
  MISC_FEATURE (1)..(9): D enantiomer
PNDPNDPND

SEQ ID NO: 73 - protein, 16 aa, Artificial Sequence
  Synthetic
  MISC_FEATURE (1)..(1): D enantiomer
  MOD_RES (2)..(2): Aib
  MISC_FEATURE (3)..(3): D enantiomer
  MISC_FEATURE (5)..(5): D enantiomer
  MOD_RES (6)..(6): Aib
  MISC_FEATURE (7)..(7): D enantiomer
  MISC_FEATURE (9)..(9): D enantiomer
  MOD_RES (10)..(10): Aib
  MISC_FEATURE (11)..(11): D enantiomer
  MISC_FEATURE (13)..(13): D enantiomer
  MOD_RES (14)..(14): Aib
  MISC_FEATURE (15)..(15): D enantiomer
DXYKDXYKDXYKDXYK

SEQ ID NO: 74 - protein, 16 aa, Artificial Sequence
  Synthetic
  MOD_RES (2)..(2): Aib
  MISC_FEATURE (4)..(4): D enantiomer
  MOD_RES (6)..(6): Aib
  MISC_FEATURE (8)..(8): D enantiomer
  MOD_RES (10)..(10): Aib
  MISC_FEATURE (12)..(12): D enantiomer
  MOD_RES (14)..(14): Aib
  MISC_FEATURE (16)..(16): D enantiomer
DXYKDXYKDXYKDXYK

SEQ ID NO: 75 - protein, 16 aa, Artificial Sequence
  Synthetic
  MISC_FEATURE (1)..(2): D enantiomer
  MISC_FEATURE (5)..(6): D enantiomer
  MISC_FEATURE (9)..(10): D enantiomer
  MISC_FEATURE (13)..(14): D enantiomer
ETPKETPKETPKETPK

SEQ ID NO: 76 - protein, 16 aa, Artificial Sequence
  Synthetic
  MISC_FEATURE (3)..(4): D enantiomer
  MISC_FEATURE (7)..(8): D enantiomer
  MISC_FEATURE (11)..(12): D enantiomer
  MISC_FEATURE (15)..(16): D enantiomer
ETPKETPKETPKETPK

SEQ ID NO: 77 - protein, 15 aa, Artificial Sequence
  Synthetic
PKDPKDPKDPKDPKD

SEQ ID NO: 78 - protein, 15 aa, Artificial Sequence
  Synthetic
  MISC_FEATURE (1)..(15): D enantiomer
PKDPKDPKDPKDPKD

SEQ ID NO: 79 - protein, 15 aa, Artificial Sequence
  Synthetic
  MISC_FEATURE (1)..(1): D enantiomer
  MISC_FEATURE (3)..(4): D enantiomer
  MISC_FEATURE (6)..(7): D enantiomer
  MISC_FEATURE (9)..(10): D enantiomer
  MISC_FEATURE (12)..(13): D enantiomer
  MISC_FEATURE (15)..(15): D enantiomer
PRDPRDPRDPRDPRD

SEQ ID NO: 80 - protein, 15 aa, Artificial Sequence
  Synthetic
  MISC_FEATURE (2)..(2): D enantiomer
  MISC_FEATURE (5)..(5): D enantiomer
  MISC_FEATURE (8)..(8): D enantiomer
  MISC_FEATURE (11)..(11): D enantiomer
  MISC_FEATURE (14)..(14): D enantiomer
PRDPRDPRDPRDPRD

SEQ ID NO: 81 - protein, 15 aa, Artificial Sequence
  Synthetic
HKDHKDHKDHKDHKD

SEQ ID NO: 82 - protein, 15 aa, Artificial Sequence
  Synthetic
  MISC_FEATURE (1)..(15): D enantiomer
HKDHKDHKDHKDHKD

SEQ ID NO: 83 - protein, 15 aa, Artificial Sequence
  Synthetic
  MISC_FEATURE (1)..(2): D enantiomer
  MISC_FEATURE (4)..(5): D enantiomer
  MISC_FEATURE (7)..(8): D enantiomer
  MISC_FEATURE (10)..(11): D enantiomer
  MISC_FEATURE (13)..(14): D enantiomer
DLRDLRDLRDLRDLR

SEQ ID NO: 84 - protein, 15 aa, Artificial Sequence
  Synthetic
  MISC_FEATURE (3)..(3): D enantiomer
  MISC_FEATURE (6)..(6): D enantiomer
  MISC_FEATURE (9)..(9): D enantiomer
  MISC_FEATURE (12)..(12): D enantiomer
  MISC_FEATURE (15)..(15): D enantiomer
DLRDLRDLRDLRDLR

SEQ ID NO: 85 - protein, 15 aa, Artificial Sequence
  Synthetic
  MISC_FEATURE (1)..(1): D enantiomer
  MISC_FEATURE (3)..(4): D enantiomer
  MISC_FEATURE (6)..(7): D enantiomer
  MISC_FEATURE (9)..(10): D enantiomer
  MISC_FEATURE (12)..(13): D enantiomer
  MISC_FEATURE (15)..(15): D enantiomer
VRDVRDVRDVRDVRD

SEQ ID NO: 86 - protein, 15 aa, Artificial Sequence
  Synthetic
  MISC_FEATURE (2)..(2): D enantiomer
  MISC_FEATURE (5)..(5): D enantiomer
  MISC_FEATURE (8)..(8): D enantiomer
  MISC_FEATURE (11)..(11): D enantiomer
  MISC_FEATURE (14)..(14): D enantiomer
VRDVRDVRDVRDVRD

SEQ ID NO: 87 - protein, 20 aa, Artificial Sequence
  Synthetic
  MISC_FEATURE (1)..(1): D enantiomer
  MISC_FEATURE (3)..(3): D enantiomer
  MISC_FEATURE (5)..(5): D enantiomer
  MISC_FEATURE (7)..(7): D enantiomer
  MISC_FEATURE (9)..(9): D enantiomer
  MISC_FEATURE (11)..(11): D enantiomer
  MISC_FEATURE (13)..(13): D enantiomer
  MISC_FEATURE (15)..(15): D enantiomer
  MISC_FEATURE (17)..(17): D enantiomer
  MISC_FEATURE (19)..(19): D enantiomer
DKETDKETDKETDKETDKET

SEQ ID NO: 88 - protein, 20 aa, Artificial Sequence
  Synthetic
  MISC_FEATURE (2)..(2): D enantiomer
  MISC_FEATURE (4)..(4): D enantiomer
  MISC_FEATURE (6)..(6): D enantiomer
  MISC_FEATURE (8)..(8): D enantiomer
  MISC_FEATURE (10)..(10): D enantiomer
  MISC_FEATURE (12)..(12): D enantiomer
  MISC_FEATURE (14)..(14): D enantiomer
  MISC_FEATURE (16)..(16): D enantiomer
  MISC_FEATURE (18)..(18): D enantiomer
  MISC_FEATURE (20)..(20): D enantiomer
DKETDKETDKETDKETDKET

SEQ ID NO: 89 - protein, 20 aa, Artificial Sequence
  Synthetic
  MISC_FEATURE (4)..(4): D enantiomer
  MISC_FEATURE (8)..(8): D enantiomer
  MISC_FEATURE (12)..(12): D enantiomer
  MISC_FEATURE (16)..(16): D enantiomer
  MISC_FEATURE (20)..(20): D enantiomer
FTAPFTAPFTAPFTAPFTAP

SEQ ID NO: 90 - protein, 20 aa, Artificial Sequence
  Synthetic
  MISC_FEATURE (1)..(3): D enantiomer
  MISC_FEATURE (5)..(7): D enantiomer
  MISC_FEATURE (9)..(11): D enantiomer
  MISC_FEATURE (13)..(15): D enantiomer
  MISC_FEATURE (17)..(19): D enantiomer
FTAPFTAPFTAPFTAPFTAP

SEQ ID NO: 91 - protein, 24 aa, Artificial Sequence
  Synthetic
  MISC_FEATURE (3)..(4): D enantiomer
  MOD_RES (5)..(5): Aib
  MISC_FEATURE (7)..(8): D enantiomer
  MOD_RES (11)..(11): Aib
  MISC_FEATURE (12)..(12): D enantiomer
  MISC_FEATURE (15)..(16): D enantiomer
  MOD_RES (17)..(17): Aib
  MISC_FEATURE (19)..(20): D enantiomer
  MOD_RES (23)..(23): Aib
  MISC_FEATURE (24)..(24): D enantiomer
KLQEXHKLQEXHKLQEXHKLQEXH